(19) United States
(12) Patent Application Publication
(10) Pub. No.: US 2025/0320509 A1
(43) Pub. Date: Oct. 16, 2025

(54) COMPOSITIONS AND METHODS FOR TREATING WILSON'S DISEASE

(71) Applicants: THE JOHNS HOPKINS UNIVERSITY, Baltimore, MD (US); UNIVERSITY OF MARYLAND MEDICAL SYSTEM, Baltimore, MD (US)

(72) Inventors: Svetlana Lutsenko, Baltimore, MD (US); Robert L. Kruse, Baltimore, MD (US); Yuting Huang, Baltimore, MD (US)

(21) Appl. No.: 18/868,037

(22) PCT Filed: May 23, 2023
(86) PCT No.: PCT/US2023/067345

§ 371 Date: Nov. 21, 2024

Related U.S. Application Data
(60) Provisional application No. 63/344,674, filed on May 23, 2022.

(51) Int. Cl. C12N 15/62; A61K 9/00; A61K 31/7088; A61K 48/00; A61P 1/16; C12N 5/10; C12N 7/00; C12N 15/85

(52) U.S. Cl. CPC C12N 15/62; A61K 9/0019; A61K 31/7088; A61K 48/0058; A61P 1/16; C12N 5/10; C12N 7/00; C12N 15/85; C12N 2795/00043; C12N 2795/00045; C12N 2795/10041

(57) ABSTRACT

Disclosed herein are compositions and methods that can be used to treat subjects with Wilson's disease. The compositions can comprise nucleic acid constructs comprising a liver-specific promoter and a nucleic acid sequence encoding the ATP73 polypeptide, a variant of the ATP73 polypeptide or a polypeptide having at least 90% identity. The compositions can be formulated for hydrodynamic gene delivery into the liver using DNA vectors.

### CROSS REFERENCE TO RELATED APPLICATIONS

This application claims the benefit of U.S. Provisional Application No. 63/344,674, filed on May 23, 2022. The content of this earlier filed application is hereby incorporated by reference herein in its entirety.

### INCORPORATION OF THE SEQUENCE LISTING

The present application contains a sequence listing that is submitted concurrent with the filing of this application, containing the file name “36406_0026P1_SL.xml” which is 20,480 bytes in size, created on May 19, 2023, and is herein incorporated by reference in its entirety.

### BACKGROUND

Wilson disease (WD) is a monogenic liver disease that results in the buildup of toxic levels of copper in different tissues, primarily affecting the liver and brain (Członkowska A, et al. Wilson disease. Nat Rev Dis Primers. 2018; 4:21-20). WD is caused by various mutations in ATP7B, which codes for a copper transporting transmembrane protein. The WD-causing mutations in ATP7B disrupt protein stability, intracellular localization, and copper transporting function. WD is a autosomal recessive disorder, and the commonly observed compound heterozygous mutations produce a broad spectrum of disease-onset and manifestations (Członkowska A, et al. Wilson disease. Nat Rev Dis Primers. 2018; 4:21-20). The liver disease can eventually progress to cirrhosis and liver failure, while the brain toxicity can result in neuropsychiatric symptoms. Treatments for WD include penicillamine and trientine, which are copper chelating agents that facilitates copper removal from the body, reducing tissue damage. Penicillamine can have significant toxicities resulting in poor compliance among WD patients (Maselbas W, et al. BMC Neurol. 2019; 19:278-6). Lack of compliance can lead to ongoing copper toxicities with patients continuing to progress in disease pathology. Thus, a need for alternative treatment strategies are needed.

### SUMMARY

Disclosed herein are nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence.

Disclosed herein are vectors comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence.

Disclosed herein are cells comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence or vectors comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence.

Disclosed herein are pharmaceutical compositions comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; cells comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or vectors comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence.

Disclosed herein are methods of delivering a nucleic acid constructs to a subject, the methods comprising administering to the subject an effective amount of a pharmaceutical composition comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or vectors comprising nucleic acid constructs comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence.

Disclosed herein are methods of treating Wilson's disease in a subject, the methods comprising: administering a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; a vector comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or a pharmaceutical composition comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence to the subject.

Disclosed herein are methods of reducing liver injury in a subject with Wilson's disease, the method comprising: administering a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; a vector comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or a pharmaceutical composition comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence to the subject.

Disclosed herein are methods of reducing hepatic copper levels in a subject with Wilson's disease, the method comprising: administering a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; a vector comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or a pharmaceutical composition comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence to the subject.

Disclosed herein are methods of reducing ALT, AST, or LDH in a subject with Wilson's disease, the method comprising: administering a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; a vector comprising a nucleic acid construct comprising: a) a promoter; b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence; or a pharmaceutical composition comprising a nucleic acid construct comprising: a) a promoter;
b) a 5′ untranslated region (5′UTR); c) a nucleic acid sequence encoding the ATP73 polypeptide, a variant of the ATP73 polypeptide or a polypeptide having at least 90% identity to SEQ ID NO: 5; d) a 3′ untranslated region (3′UTR); and e) a polyadenylation sequence to the subject.

### BRIEF DESCRIPTION OF THE DRAWINGS

FIGS. 1A-D show the construction and functional validation of plasmid expressing hATP7B with C9-tag. FIG. 1A show a C9-tag, corresponding to the carboxy-terminal 9 amino acids of bovine rhodopsin was added to the carboxy-terminus of human ATP7B located in the intracellular surface. FIG. 1B shows the gene, ATP7B,C9, was cloned into a plasmid DNA vector, pT-LP1, with a liver-specific LP1 promoter driving expression between the terminal repeats of piggyBac transposon, which facilitate integration. SV40 polyadenylation sequence is not shown. Total pDNA size is 8.6 KB, including the bacterial backbone. FIG. 1C shows the intracellular movement of hATP7B,C9 in response to different copper levels (basal, low, and high) inside cells was examined. In cells lacking native ATP7B expression, antibody staining for ATP7B and C9 tag (1D4 antibody) efficiently co-localized, with ATP7B,C9 moving out of the trans-golgi network (TGN) in response to high copper levels. FIG. 1D shows ATP7B,C9 copper transporting activity was examined in cells in cells lacking native ATP7B expression. Co-transfection of ATP7B,C9 or an inactive ATP7B (D1027A) and a plasmid encoding copper-dependent tyrosinase is performed, with successful eumelanin formation (black pigment) in cells where copper can be transported into the TGN and vesicles.

FIGS. 2A-E shows that hydrodynamic tail vein injection of pT-LP1-hATP7B,C9 results in reduction of liver injury and hepatic copper content in WD mice. FIG. 2A shows the experimental plan, wherein female WD mice were hydrodynamically injected with 1 μg of pT-LP1-hATP7B,C9 and 1 μg pCMV-hyPBase, the latter for integration. Heterozygous mice were also injected as a control, and the mice were bled and harvested at 20 weeks for further analysis. Serum obtained at 20 weeks for un-injected heterozygous mice, un-injected ATP7B KO mice, and treated ATP7B KO mice were compared for (FIG. 2B) ALT, (FIG. 2C) AST, and (FIG. 2D) LDH. FIG. 2E shows the hepatic copper content that was measured in the same mice, with significant reduction in the treatment group. Statistical significance was calculated with unpaired, parametric t-tests, * p<0.05, ** p<0.005, *** p<0.0005.

FIGS. 3A-C show that hydrodynamic tail vein injection achieves transfection of hepatocytes with ATP7B, C9 without significant expansion over time. FIG. 3A shows that immunohistochemical staining for ATP7B,C9 for treated WD mice reveals positive hepatocytes with variable morphology and staining intensity. FIG. 3B shows the quantification of ATP7B,C9 transfection in KO mice and heterozygous mice by percent area stained, measured six weeks after injection (8 random 20× fields measured, n=2 mice each group). FIG. 3C shows H&E staining for female heterozygous, KO untreated, and KO treated mice with minimal differences.

FIGS. 4A-E show that biliary hydrodynamic injection can successfully mediate hATP7B,C9 expression in pig liver. FIG. 4A shows ERCP can be used to access the common hepatic duct, with fluoroscopy verifying catheter position and branching into the liver prior to injection. FIG. 4B shows that harvested pig liver has no abnormalities from injection and no rupture of bile ducts. Sampling of the right lateral, right medial, left medial, and later lateral liver lobes was conducted. FIG. 4C shows that immunohistochemistry for C9-tag in pig liver reveals abundant hATP7B,C9 positive hepatocytes, which were located across the three zones. Rare liver lobules had transfection that exceeded over 80% of hepatocytes. FIG. 4D shows immunofluorescent staining for ATP7B,C9 in pigs demonstrating localization of in ATP7B,C9 inside pig hepatocytes. This pattern was similar to ATP7B staining for native pig ATP7B. FIG. 4E shows the quantification of immunostaining in pigs dosed at 9 mg pDNA and 5 mg pDNA, respectively. Percent area was calculated among liver lobules, with 8 liver lobules per pig averaged.

FIGS. 5A-E show serum chemistries reported for WD mice treated with 1 μg of pT-LP1-hATP7B,C9 by hydrodynamic injection. Mice were bled at 20 weeks age, and six weeks post-DNA injection. ATP7B KO mice were treated with 1 μg of pT-LP1-hATP7B,C9, while control groups of untreated heterozygous and KO mice were also analyzed. Albumin (FIG. 5A), Alkaline phosphatase (FIG. 5B), total bilirubin (FIG. 5C), total protein (FIG. 5D), and glucose (FIG. 5E) are presented. Statistical significance was calculated with unpaired, parametric t-tests, * p<0.05, ** p<0.005, *** p<0.0005, n.s. non-significant.

FIGS. 6A-D show that hydrodynamic injection of 25 μg of pT-LP1-hATP7B,C9 into WD mice did not yield improvements in liver injury. FIG. 6A shows the experimental plan, wherein male and female WD mice were hydrodynamically injected with 25 μg of pT-LP1-hATP7B,C9 and 10 μg pCMV-hyPBase, the latter for integration. Mice were injected prior to WD liver phenotype development, and mice were harvested at 20 weeks for further analysis. The same female heterozygous and KO mice depicted in FIG. 2 are presented here as control groups for comparison. Serum chemistries for ALT (FIG. 6B), AST (FIG. 6C), and LDH (FIG. 6D) are presented. Statistical significance was calculated with unpaired, parametric t-tests, * p<0.05, ** p<0.005, *** p<0.0005, n.s. non-significant.

FIGS. 7A-C show that hydrodynamic injection of 25 μg of pT-LP1-hATP7B,C9 mediates high-level expression in mouse liver, but expression is lost over time. FIG. 7A shows that hydrodynamic injection of 25 μg of pT-LP1-hATP7B,C9 and 10 μg pCMV-hyPBase mediates high-level expression and transfection efficiency in mice 4 days after transfection. FIG. 7B shows quantification of the percent area stained for the mice dosed with 25 μg of pT-LP1-hATP7B,C9, four days post-injection. 8 random 20× fields, n=2 mice presented. FIG. 7C shows mice that are 12 weeks post-hydrodynamic injection of 25 μg of pT-LP1-hATP7B,C9 and 10 μg pCMV-hyPBase as examined by immunofluorescence for ATP7B,C9 expression using the 1D4 antibody and anti-mouse FITC secondary. Mice harvested 4 days post-injection served as a control.

FIGS. 8A-B show that hydrodynamic injection of 5 μg of pT-LP1-hATP7B,C9 into WD mice did not yield improvements in liver injury. FIG. 8A shows the experimental plan, wherein male and female WD mice were hydrodynamically injected with 5 μg of pT-LP1-hATP7B,C9 and 5 μg pCMV-hyPBase, the latter for integration. Mice were injected prior to WD liver phenotype development, and mice were harvested at 20 weeks for further analysis. The same female heterozygous and KO mice depicted in FIG. 2 are presented here as control groups for comparison. Serum chemistries for ALT (FIG. 8B), AST (FIG. 8C), and LDH (FIG. 8D) are presented. Statistical significance was calculated with unpaired, parametric t-tests, * p<0.05, ** p<0.005, *** p<0.0005, n.s. non-significant.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for Wilson's disease, such as, for example, prior to the administering step.

As used herein, the term “comprising” can include the aspects “consisting of” and “consisting essentially of.”

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be CDNA, DNA, or mRNA.

The term “sequence of interest” or “gene of interest” can also mean a nucleic acid sequence that is partly or entirely complementary to an endogenous gene of the cell into which it is introduced.

A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence (e.g., a therapeutic protein), that is expressed from a sequence of interest or gene of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the(S) configuration. In some aspects, the amino acids are in the(S) or L-configuration. If non-naturally occurring side chains are used, non-amino acid substituents may be used, for example to prevent or retard in vivo degradation.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Although the foregoing invention has been described in some detail by way of illustration and example for purposes of clarity of understanding, certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Wilson's disease is caused by mutations in the ATP7B gene leading to decreased function of the protein in transporting copper. The lack of normal ATP7B function leads to the buildup of copper in the liver, brain and other tissues, eventually leading to organ damage in these tissues and various clinical signs and symptoms.

Previous attempts at gene therapy for Wilson's disease have focused the delivery of ATP7B using adeno-associated virus (AAV) vectors. AAV vectors have size limitations of around 4.7-8 kb in total. This size limitation makes it challenging to use the platform as an ATP7B delivery vehicle, given that ATP7B has a vector size of 4.4 kb. While ATP7B is expressed in many different tissues, the replacement of ATP7B gene has primarily focused on the liver. The liver plays a central role in the whole-body copper homeostasis and ATP7B is important for this liver function: ATP7B facilitates the delivery of copper to ceruloplasmin (the major copper containing protein in a serum) and exports excess copper into bile. Liver transplants are able to cure WD in patients developing liver failure, and recent clinical studies suggest that they may improve neuropsychiatric symptoms. (3) Liver transplants are, however, a risky procedure with numerous comorbidities and require the lifelong maintenance of immunosuppression to ensure graft survival. The limited amount of available organs and these potential toxicities reduce the use of liver transplantation for WD treatment.

Gene therapy could solve many issues with liver transplant by directly delivering the gene into the patient's hepatocytes without the need for long-term immunosuppression. Gene therapy has been explored and showed promise in WD mouse models. Using adeno-associated viruses (AAV) as the delivery vehicle for ATP7B, significant improvements in liver enzyme function and reductions in copper in the liver and other tissues were achieved (Murillo O, et al. Journal of Hepatology. 2016; 64:419-426; and Murillo O, et al. Hepatology. 2019; 70:108-126). However, a chief problem using AAV has been the size limitation of 4.8 kB of the vector, while the ATP7B gene itself is 4.4 KB leaving not enough room for the gene expression elements and AAV ITR's. Investigators have utilized truncated forms of ATP7B with several of the metal binding domains deleted (Leng Y, et al. Human Gene Therapy. 2019; 30:1494-1504), but these truncated forms may have altered protein function and regulation compared to the full-length protein. In order to deliver full length ATP7B with regulatory elements, non-viral approaches without size restriction could be utilized. Hydrodynamic gene delivery can deliver naked plasmid DNA (pDNA) directly into hepatocytes but has not previously been explored to deliver ATP7B for WD gene therapy.

Disclosed herein are compositions that can be used to non-viral hydrodynamic deliver the full-length ATP7B gene to hepatocytes and methods of using the same. In an aspect, a transposon system was used to mediate integration into the liver genome for expansion of hepatocytes. Seeking to translate hATP7B gene delivery into large animals, the disclosed, the disclosed compositions and non-viral gene delivery systems disclosed herein can be modeled by biliary hydrodynamic injection into pig liver paving the way for clinical translation.

The nucleic acid constructs and DNA vectors described herein for expressing ATP7B in human subjects provide several important improvements as described herein.

In some aspects, the methods described herein comprise the delivery of a nucleic acid construct into hepatocytes of a subject with Wilson's disease, with the DNA molecule entering into cell and eventually the nucleus of hepatocytes yielding expression of the ATP73 polypeptide, a variant of the ATP73 polypeptide or a polypeptide having at least 90% identity to SEQ ID NO: 5. In some aspects, DNA molecule or nucleic acid construct can be a circular DNA molecule, a plasmid DNA molecule, or derived from a plasmid DNA molecule. In some aspects, the DNA molecule or nucleic acid construct can be a linear DNA molecule comprising covalently closed ends having the bacterial sequences removed from the vector.

In some aspects, the nucleic acid construct s disclosed herein can contain a bacterial backbone that can be miniature in size compared to the most commonly used plasmid cloning vectors in the art. In some aspects, the bacterial backbone sizes can be optimally below one kilobase in total length, as defined by nucleotide length between the promoter region and the polyadenylation sequence, both representing non-mammalian elements. In some aspects, the DNA vector, can include using bacterial origins that are reduced in size such as the R6K origin, or a miniature pUC origin. Full-length bacterial origins can also be used in combination with smaller resistance genes in order to meet this length requirement. In some aspects, the bacterial sequence can be less than 750 bps or less than 500 bps in the episomal DNA vector.

Several elements can be utilized for the selection of plasmids as the bacterial hosts for propagation. These include but are not limited to antibiotic resistance genes (e.g., s ampicillin resistance gene, kanamycin resistance gene, zeocin resistance gene, and chloramphenicol resistance gene). Alternative systems of selection include the RNA-OUT system suppressing target expression of toxic gene, providing tRNA suppressor genes (pFAR4) system, or ccdB/ccdA.

In some aspects, the nucleic acid constructs and vectors disclosed herein have no distinct size limitations for the DNA vector encoding ATP7B. In some aspects, DNA vectors comprising the nucleic acid constructs can preferably be as small as possible to increase delivery efficiency and/or yield from DNA manufacturing. For example, as demonstrated herein, delivery of a 8.4 kb size of plasmid DNA into the livers of mice and pigs by hydrodynamic gene delivery.

The promoter sequence can be an important element to designing any gene therapy. As described herein, AAV gene therapy has significant size limitations, which has focused the AAV gene therapy field on small, short promoters that are expected to have high expression activity. Unfortunately, these smaller promoters are largely unregulated with respect to the gene being encoded, lacking the native gene-specific regulation of many genes to sense extracellular and intracellular conditions.

With regard to the nucleic acid constructs and uses of the same ATP7B as described herein, an expected and surprising high-level, unregulated expression was observed. For example, when a plasmid encoding a liver-specific promoter based on a chimeric enhancer-promoter combination of APO HCR and alpha-1 antitrypsin (AAT) promoter is delivered, it leads to efficient expression of ATP7B, although this expression was found to be toxic to the hepatocytes when too many vector copies are delivered into cells, resulting in the loss of ATP7B expression. Such overexpression toxicity has not been reported in the literature and is therefore surprising. To address this limitation, the nucleic acid constructs and gene delivery methods disclosed herein provides solutions to these limitations.

One solution is that the method of gene therapy can be adjusted, wherein lower concentrations of plasmid DNA encoding ATP7B under the direction of a high-level liver-specific promoter can be performed. This can serve to titrate the DNA dose and subsequently the amount of ATP7B expression inside the cell. While fewer cells may receive the ATP7B gene, the amount of plasmid DNA distributed among the liver cells is heterogeneous.

In some aspects, ATP7B can be expressed using promoters that are regulated by the amount of copper and/or metals in the environment. Examples of said promoters include but are not limited to the native ATP7B promoter itself, as well as the metallothionein promoter. In some aspects, hybrid promoters can be used such that the core promoter can be a ubiquitous liver-specific promoter such as alpha-one antitrypsin (AAT) promoter. In some aspects, the enhancer element that upregulates promoter expression can be based on a metal responsive element (MRE) found in the ATP7B or metallothionein promoters. In some aspects, the native ATP7B promoter without any sequence alterations can be utilized up to 1500, 1200 or 1000 bp's ahead of the native ATP7B. Since the exact ATP7B promoter is undefined, multiple different sequence lengths as listed can also be utilized. In some aspects, at least 500 base pairs of sequence ahead of the native ATG start codon in the human genome of ATP7B can be utilized. In some aspects, at least 1000 base pairs upstream of the ATG sequence in the human genome of ATP7B can be used. In some aspects, a synthetic promoter with multiple repeats of MRE elements can be utilized to drive ATP7B expression.

In some aspects, a core region of the ATP7B promoter can be utilized, which encodes sequences containing the positive regulatory elements of ATP7B promoter (−800 bp ahead of the native start codon of ATP7B gene) resulting in higher expression. In some aspects, the negative regulatory regions of the ATP7B promoter can be utilized.

In some aspects, the native ATP7B promoter can be further modified. In some aspects, the modifications to the native ATP7B promoter can include the addition of enhancer elements to the ATP7B promoter to augment gene expression while maintaining regulation. While ATP7B promoters have the benefit of maintaining natural regulatory process to sense copper levels, the limitation is that the transcript level is relatively low compared to other liver-specific promoters used in gene therapy. This is particularly important when using episomal plasmid DNA vectors, which have generally lower expression of mRNA compared to the same gene on the host chromosome. The ATP7B promoter is significantly weaker versus the native alpha-1 antitrypsin (AAT) promoter by comparison, which corresponds to the relative amounts of each proteins that are required for normal human physiology.

In some aspects, liver-specific enhancer elements including but not limited to APO HCR and ApoE can be added to the 5′ end of the ATP7B promoter. In some aspects, enhancers derived from different viruses including but not limited to simian virus 40 and hepatitis B virus can also be added, which have general properties of increasing promoter strength, while maintaining the specificity of cell-type expression and regulation. Examples of these enhancer elements include but are not limited to SV40 enhancer, HBV enhancer I, and HBV enhancer II. In some aspects, negative regulatory elements can be removed from the ATP7B promoter in the region −1200 to −800 and replaced with liver-specific or viral enhancers to enhance the expression ATP7B.

In some aspects, a liver specific core promoter can be utilized, but the enhancer element can be derived from metal regulatory elements (MRE's) that are governed by transcription factors, which sense the concentrations of different metals inside the cell, including copper. A DNA sequence that includes multiple different copies of these metal regulatable elements (MRE's) can be included 5′ to a core liver specific promoter such as alpha-1 antitrypsin, in order to achieve effective enhancement of gene expression in response to the levels of metal inside the cell.

In some aspects, other promoters can be used in the nucleic acid constructs described herein. For example, metallothionein is an important protein that helps sequester different metal ions in the body. The metallothionein promoter also responds to increased levels of metal ions in order to facilitate more production of metallothionein protein to sequester the metal ions. As such, the metallothionein promoter is an alternative to regulate ATP7B expression inside hepatocytes.

Other elements of the nucleic acid constructs and DNA vectors are also important for optimizing expression of the ATP7B gene, particularly in the situation where the promoter is relatively weak and is being regulated by copper status. In some aspects, other elements can be utilized to counteract the relatively weaker expression from plasmid DNA in order to achieve sufficiently high ATP7B levels that are still regulated by copper levels. In some aspects, the 5′ UTR can have an intron introduced into it, in order to increase mRNA export from the nucleus an ultimately expression in the cytoplasm. These introns can be non-native to the 5′ UTR of human ATP7B. In some aspects, the metallothionein or alpha-1 antitrypsin promoters can be used. In some aspects, the nucleic acid constructs and DNA vectors can include miniature intronic elements from viral sequences (e.g., SV40 or MVM).

In some aspects, particularly when the native ATP7B promoter is utilized, the native ATP7B 5′ UTR can be utilized in the vector, such that introns can be introduced into the coding sequence of the ATP7B protein, where they otherwise do not naturally exist. In some aspects, at least one intron can be introduced. In some aspects, two or more introns can be introduced.

In some aspects, a 3′ UTR can be added downstream of the ATP7B coding sequence that would lead to the enhancement of expression. The 3′ UTR can function to increase the half-life of mRNA inside the cell's cytoplasm, as well as by enhancing translational potency of a given mRNA molecule. This would effectively increase ATP7B expression without interrupting its regulation from its promoter element. Examples of 3′ UTR elements that can be used for this purpose include but are not limited to the human alpha and beta globin gene 3′ UTR regions.

A disadvantage of AAV vectors and plasmid DNA vectors is their episomal status. Because DNA vectors are not integrated into the genome, they can be lost overtime with the division of hepatocytes. In aspects, sequences can be added to the nucleic acid constructs and DNA vectors that can allow for replication during mitosis in cells. These sequences can be derived from scaffold matrix attachment regions (S/MAR) elements, which could be included in the nucleic acid constructs or DNA vectors to facilitate replication. In some aspects, S/MAR elements can be placed 3′ region to or 3′ UTR of the ATP7B gene cassette. Given the liver damage and hepatocyte turnover in patients with Wilson's disease, there is a possibility of a proliferative advantage of hepatocytes expressing ATP7B given that they will be protected from copper overload. Indeed, this has been observed in different cell transplantation studies of Wilson's disease (Mol Ther. 2001 March; 3(3):302-9). The S/MAR sequence on the vector can facilitate effective replication of the episomal DNA vector with hepatocyte mitosis, such that hepatocytes expressing ATP7B can be protected from copper toxicity, and thus, the percentage of positive hepatocytes can increase within the liver over time.

In some aspects, the nucleic acid constructs can comprise a DNA sequence directing polyadenylation of the mRNA transcripts generated, as is customary in most expression vectors. Examples of polyadenylation sequences include but are not limited to a SV40 polyadenylation sequence, a human growth hormone polyadenylation sequence, a bovine growth hormone polyadenylation sequence, and a rabbit beta-globin polyadenylation sequence.

In some aspects, the coding sequence of ATP7B is a feature of the nucleic acid construct, and thus, the DNA vector, which can improve the vector potency and resultant therapeutic activity. In some aspects, the full length ATP7B gene can be utilized. The coding sequence can be interrupted with additional introns in order to increase expression. In some aspects, a small C-terminal protein tag can be added to distinguish between endogenous mutant ATP7B and the vector delivered wild-type ATP7B transgene. The C-terminal tag should not disrupt the native ATP7B function and/or trafficking within the cell. Examples of C-terminal tags include but are not limited to the C9-tag derived from the C-terminal 9 amino acids from the human rhodopsin protein, and the c-myc tag. The exact DNA sequence of ATP7B can be the same as that encoded in the human genome. In some aspects, the DNA coding sequence can be codon optimized to increase the levels of usage of common DNA codons in the human cells, such that the overall protein expression is increased. In some aspects, the DNA sequence can be completely different from the native human ATP7B sequence, but the protein coded can be the same wild-type ATP7B gene.

In some aspects, different small nucleotide polymorphisms (SNPs) can be present in the coding sequence of ATP7B. ATP7B is a protein (1,465 amino acids), in which there exists no canonical truly wild-type sequence. Different SNP's exist in the human population. Depending on the combination of SNP's utilized, there will be different levels of ATP7B activity in its copper transporting properties. In some aspects, SNP's existing at K832 and R952 can be incorporated to increase the copper transport activity of ATP7B, leading to higher overall reduction in copper levels in spite of lower amounts of ATP7B protein.

The nucleic acid constructs and vectors disclosed herein can be delivered through hydrodynamic injection. Routes of hydrodynamic injection include vascular and biliary routes. In either route, the nucleic acid constructs or DNA vectors can be dissolved in a pharmaceutically acceptable solution, such as normal saline, phosphate buffered solution, lactate ringer's solution, or dextrose solution. Optimal pressure can be obtained that creates pores in cell membranes in order to deliver the DNA vector inside cells. In some aspects, the nucleic acid constructs or DNA vectors can be encapsulated within a lipid particle or a lipid nanoparticle to facilitate DNA protection and cell uptake.

### Compositions

Nucleic acid constructs. Disclosed herein are nucleic acid constructs. Any combination of the nucleic acid construct disclosed herein can be present in a single nucleic acid construct. Table 1 provides sequences that can be incorporated into the disclosed nucleic acid constructs.

Described herein are nucleic acid constructs comprising: a promoter; a 5′ untranslated region (5′UTR); a nucleic acid sequence encoding the ATP73 polypeptide, a variant of the ATP73 polypeptide or a polypeptide having at least 90% identity to SEQ ID NO: 5; a 3′ untranslated region (3′UTR); and a polyadenylation sequence.

Also disclosed herein are vectors comprising nucleic acid constructs. In some aspects, the nucleic constructs can comprise a promoter; a 5′ untranslated region (5′UTR); a nucleic acid sequence encoding the ATP73 polypeptide, a variant of the ATP73 polypeptide or a polypeptide having at least 90% identity to SEQ ID NO: 5; a 3′ untranslated region (3′UTR); and a polyadenylation sequence.

In some aspects, the nucleic acid constructs can further comprise a bacterial origin of replication and/or a selection system. In some aspects, the bacterial origin of replication can comprise a pUC origin, R6K origin, or a miniaturized pUC origin. In some aspects, the selection system can comprise an RNA-OUT, tRNA, cccdB/cccdA, or an antibiotic resistance gene. In some aspects, the antibiotic resistance gene can be ampicillin, kanamycin, or zeocin. In some aspects, the bacterial origin or backbone can comprise various elements as disclosed herein that can be less than 1 kb in size or less than 500 bp in size to avoid silencing of the DNA vector.

ATP73 gene/ATP73 polypeptide. In some aspects, the nucleic acid encoding ATP73 polypeptide, a variant of the ATP73 polypeptide or a polypeptide having at least 90% identity to SEQ ID NO: 5 can be the ATP73 human gene sequence or a variant thereof. The ATP73 human gene sequence corresponds to NCBI reference sequence: NC_000013.11.

In some aspects, the nucleic acid sequence encoding the ATP73 polypeptide can be a cDNA encoding full-length ATP73 protein. In some aspects, the nucleic acid sequence encoding the ATP73 polypeptide can comprise one or more introns. In some aspects, the one or more introns introduced into the cDNA sequence can increase expression of the ATP73 protein. In some aspects, the nucleic acid sequence encodes a variant of the ATP73 polypeptide comprising a polymorphism at position K832, R952, or a combination thereof. In some aspects, one or more sequence variants at positions, K832, R952, or a combination thereof can increase copper transporting activity. In some aspects, the nucleic acid sequence encoding the ATP73 polypeptide can be codon optimized. In some aspects, codon optimization can increase expression inhuman cells.

In some aspects, the nucleic acid sequence encoding the ATP73 polypeptide can further comprise a protein tag or a detectable moiety. In some aspects, the protein tag or the detectable moiety can be at the C-terminus. In some aspects, the protein tag or a detectable moiety can be used to track delivery of nucleic acid sequence encoding the ATP73 polypeptide, a variant of the ATP73 polypeptide or a polypeptide having at least 90% identity to SEQ ID NO: 5 into cells without effecting protein expression. Examples of protein tags and detectable moieties include but are not limited to fluorescein for fluorescence, HA tag, Gst-tag, EGFP-tag, FLAG™ tag or biotin. In some aspects, the protein tag or detectable moiety can be a C9 tag or a c-myc tag. In some aspects, the C9 tag can 9 terminal acid amino residues of the bovine rhodopsin gene (TETSQVAPA; SEQ ID NO: 11).

Vectors. Disclosed herein are vectors comprising any of the nucleic acid constructs described herein. Vectors comprising nucleic acids or polynucleotides as described herein are also provided. As used herein, a “vector” refers to a carrier molecule into which another DNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids (e.g., bacteriophage), and artificial chromosomes (e.g., YACs). In some aspects, the vector can be a non-viral vector. In some aspects, the vector is not a virus. In some aspects, the vector can be a plasmid or a bacteriophage. In some aspects, the vector can be a double-stranded DNA. In some aspects, the double-stranded DNA can be in a circular or linear form.

In some aspects, the vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids. The term “vector” includes expression vectors and refers to a vector containing a nucleic acid sequence coding for at least part of a gene product capable of being transcribed. A variety of ways can be used to introduce an expression vector into cells. As used herein, “expression vector” is a vector that includes a regulatory region. A variety of host/expression vector combinations can be used to express the nucleic acid sequences disclosed herein. Examples of expression vectors include but are not limited to plasmids vectors derived from, for example, bacteriophages. Vectors and expression systems are commercially available and known to one skilled in the art.

The vectors disclosed herein can also include detectable label or selectable marker. Such detectable labels can include a tag sequence designed for detection (e.g., purification or localization) of an expressed polypeptide. Tag sequences or detectable labels can include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus.

The term “expression cassette” as used herein refers to a nucleic acid construct. The expression cassette can be produced either through recombinant techniques or synthetically that will result in the transcription of a certain polynucleotide sequence in a host cell. The expression cassette can be part of a plasmid or nucleic acid fragment. Generally, the expression cassette includes a polynucleotide operably linked to a promoter. In some aspects, the expression cassette can be a plasmid. The expression cassette can be adapted for expression in a specific type of host cell. The expression cassette can also comprise other components such as polyadenylation signals, enhancer elements or any other component that results in the expression of the nucleic acid constructs disclosed herein in a specific type of host cell.

Vectors include, for example, liposomes and other lipid-containing complexes, and other macromolecular complexes capable of mediating delivery of a polynucleotide to a host cell. Vectors can also comprise other components to further modulate the delivery and/or expression of the gene of interest, for example, or that otherwise provides beneficial properties to the targeted cells. A wide variety of vectors is known to those skilled in the art and is generally available. Other suitable complexes capable of mediating delivery of any of the nucleic acid constructs described herein include vaults, cell penetrating peptides and biolistic particle guns. Cell penetrating peptides are capable of transporting or translocating proteins across a plasma membrane; thus, cell penetrating peptides act as delivery vehicles. Examples include but are not limited to labels (e.g., GFP, MRI contrast agents, quantum dots).

In some aspects, the vectors (e.g., DNA vectors) disclosed herein can be produced, generated or processed in E. coli or in vitro with the TelN protelomerase enzyme to convert it into a linear DNA form and eliminate any host bacterial elements. In some aspects, the vectors (e.g., DNA vectors) disclosed herein can be produced, generated or processed in E. coli or in vitro or in a target mammalian liver with a recombination enzyme to generate a circular DNA molecule free of bacterial elements, also known as a minicircle. In some aspects, the circular DNA molecule can have two recognition sites within the vector including but not limited to LoxP, attB/attP, MRS, or FRT mediating recognition by Cre recombinase, ParA resolve, phiC31integrase, and FLP recombinase.

Promoters. In some aspects, the nucleic acid constructs disclosed herein can comprise a promoter. In some aspects, the promoter can be a cell type specific promoter. In some aspects, the promoter can be a liver-specific promoter. In some aspects, the liver-specific promoter can be alpha-1 antitrypsin, human thyroxine binding globulin, hemopexin, albumin, or HBV core promoter. In some aspects, the promoter can be the human ATP7B promoter. In some aspects, the promoter can be a portion of the human ATP7B promoter. In some aspects, the promoter can be a segment of the human ATP7B promoter. In some aspects, the segment of the human ATP7B promoter can comprise a negative regulatory element. In some aspects, the negative regulatory element can be replaced with an enhancer element. In some aspects, the promoter can maintain native gene regulation and avoid overexpression cytotoxicity. In some aspects, the promoter can be a portion or segment of the human ATP7B promoter that is capable of maintaining native gene regulation and avoiding overexpression cytotoxicity. In some aspects, the ATP7B promoter can comprise or consist of a sequence of a region that can be 1500 bp, 1000 bp, 750 bp, or 500 bp's ahead of the native start codon of the human ATP7B gene. In some aspects, nucleotides −811 to −1265 of the negative regulatory element of the ATP7B promoter can be replaced with an enhancer element. In some aspects, the promoter can be the human metallothionein promoter. In some aspects, the promoter can be the human metallothionein promoter for metal responsive transcription. In some aspects, the promoter can be a synthetic promoter. In some aspects, the synthetic promoter can comprise or consist of one or more MTF1 transcription factor binding sites. In some aspects, the synthetic promoter can comprise or consist of one or more MTF1 transcription factor binding sites to yield regulation by metals.

In some aspects, the promoter can be operatively linked to 5′UTR. In some aspects, the promoter can be operatively linked to a start codon. In some aspects, the promoter can be regulatable. In some aspects, the promoter can be constitutively active.

As used herein, the term “promoter” refers to regulatory elements, promoters, promoter enhancers, internal ribosomal entry sites (IRES) and other elements that are capable of controlling expression (e.g., transcription termination signals, including but not limited to polyadenylation signals and poly-U sequences). Promoters can direct constitutive expression. Promoters can also direct expression in a temporal-dependent manner including but not limited to cell-cycle dependent or developmental stage-dependent. Examples of promoters include but are not limited to WPRE, CMV enhancers, and SV40 enhancers. Specific gene specific promoters can be used. Such promoters allow cell specific expression or expression tied to specific pathways. Any promoter that is active in mammalian cells can be used. In some aspects, the promoter can be an inducible promoter including, but not limited to, Tet-on and Tet-off systems. Such inducible promoters can be used to control the timing of the desired expression. In some aspects, the promoter can be an inducible promoter. Examples of inducible promoters include but are not limited to tetracycline inducible system (tet); heat shock promoters and IPTG activated promoters. In some aspects, promoters can be bidirectional.

The promoter and/or enhancer can be specifically activated either by light or specific chemical events which trigger their function. Systems can be regulated by reagents such as tetracycline and dexamethasone.

In some aspects, the enhancer element can be a liver-specific enhancer element. In some aspects, the liver-specific enhancer element can be a human apolipoprotein hepatic control region, human albumin enhancer, human ApoE enhancer, or a viral enhancer. In some aspects, the viral enhancer can a SV40 enhancer, a HBV enhancer I, or a HBV enhancer II. In some aspects, the liver-specific enhancer element can comprise a metal responsive element (MRE) site. In some aspects, the MRE site can be responsive to copper. In some aspects, the liver-specific enhancer element comprises metal responsive element (MRE) sites to promote regulation by copper levels and avoid cytotoxicity of ATP7B overexpression. 5′UTR. In some aspects, the 5′UTR can comprise an intron. In some aspects, the intron can be a SV40 intron, a Minute Virus of Mice (MVM) intron, or a human growth hormone (HGH) intron. The word intron is derived from the terms intragenic region (Gilbert W (February 1978). Nature. 271 (5645): 501), and intracistron (Tonegawa S, Maxam A M, Tizard R, Bernard O, Gilbert W (March 1978). Proceedings of the National Academy of Sciences of the United States of America. 75 (3): 1485-9), that is, a segment of DNA that is located between two exons of a gene. The term intron refers to both the DNA sequence within a gene and the corresponding sequence in the unprocessed RNA transcript. As part of the RNA processing pathway, introns are removed by RNA splicing either shortly after or concurrent with transcription (Tilgner H, Knowles D G, Johnson R, Davis C A, Chakrabortty S, Djebali S, Curado J, Snyder M, Gingeras T R, Guigó R (September 2012). Genome Research. 22 (9): 1616-25). Introns are found in the genes of most organisms and many viruses. They can be located in a wide range of genes, including those that generate proteins, ribosomal RNA (rRNA), and transfer RNA (tRNA) (Roy S W, Gilbert W (March 2006). Genetics. 7 (3): 211-21).

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint, which includes an adenine nucleotide involved in lariat formation (Clancy S (2008). Nature Education. 1 (1): 31; and Black D L (June 2003). Annual Review of Biochemistry. 72 (1): 291-336). The consensus sequence for an intron (in IUPAC nucleic acid notation) is: G-G-[cut]-G-U-R-A-G-U (donor site) . . . intron sequence . . . Y-U-R-A-C (branch sequence 20-50 nucleotides upstream of acceptor site) . . . Y-rich-N-C-A-G-[cut]-G (acceptor site) (“Molecular Biology of the Cell”. 2012 Journal Citation Reports. Web of Science (Science ed.). Thomson Reuters. 2013). However, it is noted that the specific sequence of intronic splicing elements and the number of nucleotides between the branchpoint and the nearest 3′ acceptor site affect splice site selection (Taggart A J, DeSimone A M, Shih J S, Filloux M E, Fairbrother W G (June 2012). “Nature Structural & Molecular Biology. 19 (7): 719-21; and Corvelo A, Hallegger M, Smith C W, Eyras E (November 2010). Meyer (ed.). PLOS Computational Biology 6 (11)). Also, point mutations in the underlying DNA or errors during transcription can activate a cryptic splice site in part of the transcript that usually is not spliced. This results in a mature messenger RNA with a missing section of an exon. In this way, a point mutation, which might otherwise affect a single amino acid, can manifest as a deletion or truncation in the final protein.

Also described herein are intron cassettes that comprise one or more exon splicing enhancer sequences. Splicing enhancer sequences confer cell specificity during exon splicing. In some aspects, the splicing enhancer sequences are not known. For exon splicing to occur, a cell type of interest must express the specific splicing factors (e.g., proteins that bind to RNA) that can induce the splicing of a cell specific exon sequence (for example, see, Baralle and Giudice, Nat. Rev. Mol. Cell Biol. 2017, 18(7): 437-451). As long as the nucleic construct can be delivered to the target cell or cell type of interest, the splicing machinery in the target cell or cell type of interest will do all the work. In some aspects, the intron cassette can be spliced in a cell type of interest.

3′UTR. In some aspect, the 3′UTR can be alpha-hemoglobin, beta-hemoglobin, albumin, mitochondrially encoded 12S IRNA (mtRNR1), or amino-terminal enhancer of split (AES). In some aspect, the 3′UTR can be alpha-hemoglobin, beta-hemoglobin, albumin, mitochondrially encoded 12S rRNA (mtRNR1), or amino-terminal enhancer of split (AES) that is capable of stabilizing the mRNA transcript and increasing gene or protein expression. In some aspects, the 3′UTR can comprise a scaffold/matrix attachment region (S/MAR) element. In some aspects, the scaffold/matrix attachment region (S/MAR) element can allow for DNA replication with mitosis.

Polyadenylation sequence. In some aspects, the nucleic acid constructs disclosed herein can comprise a polyadenylation signal. In some aspects, the polyadenylation signal can be preceded by a 3′UTR. In some aspects, the 3′UTR can be positioned between the a nucleic acid sequence encoding the ATP73 polypeptide, a variant of the ATP73 polypeptide or a polypeptide having at least 90% identity to SEQ ID NO: 5 and the polyadenylation signal. In some aspects, the polyadenylation sequence can comprise a SV40 polyadenylation sequence, a human growth hormone polyadenylation sequence, a bovine growth hormone polyadenylation sequence, or a rabbit beta-globin polyadenylation sequence.

Start codons. In some aspects, the nucleic acid constructs disclosed herein can comprise a start codon. In some aspects, the start codon can be ATG. In some aspects, the start codon can be preceded by a 5′UTR. In some aspects, the 5′UTR can be positioned between the promoter and the start codon.

The term “codon” denotes an oligonucleotide consisting of three nucleotides that encodes a defined amino acid. Due to the degeneracy of the genetic code some amino acids are encoded by more than one codon. These different codons encoding the same amino acid have different relative usage frequencies in individual host cells. Thus, a specific amino acid can be encoded by a group of different codons. Likewise the amino acid sequence of a polypeptide can be encoded by different nucleic acids. Therefore, a specific amino acid can be encoded by a group of different codons, whereby each of these codons has a usage frequency within a given host cell.

Cells. Disclosed herein are cells comprising any of the nucleic acid constructs described herein. Also disclosed herein are cells comprising any of the vectors comprising any of the nucleic acid constructs described herein. In some aspects, the cell can be a specific cell. In some aspects, the cell can be a eukaryotic cell. In some aspects, the eukaryotic cell can be a mammalian cell. In some aspects the cell can be a specific eukaryotic cell. In some aspects, the cell can be a specific mammalian cell. In some aspects, the cell can be a cell within a subject. In some aspects, the cell can be a liver cell. In some aspects, the cell can be a diseased cell. In some aspects, the eukaryotic cell can be a diseased cell. In some aspects, the mammalian cell can be a diseased cell.

In some aspects, the nucleic acid constructs as described herein can be delivered to a cell of a subject.

TABLE 1
Sequences.

| SEQ | | |
|---|---|---|
| ID | | |
| NO: | Name | Sequence |
| 1 | Full length ATP7B | acaaggaaggccatttgcccgcaaaatttagctacactggacgggcaagtacccct |
| | promoter sequence; | acagaagagaaaacgtctgtgagcccacacgaccggctgctcacctcaacaacttg |
| | The sequence starts at | cacaggcaccagctcctttcgccggccgccatcttccgccgacccccgaactcagg |
| | −895 before | aaacgcttcactttccttttccctattggctcctgagaaagcaagccgtgctcgccccg |
| | transcription start site | cccccacgggccaattgtgcgttactattggttactggtagccgcttcccacggcctt |
| | to +175 of | ccagccaatagaatatgccgaggcgtagactagtgttcggcgtggcgcacacggct |
| | transcription. This | cccgcccccgtgggcgggacagcagtggggggttgggctgaggagggcgtggc |
| | sequence is designed | ctgtgattgacagccgtcgctccctcccteggccacctcccccactagaagccccc |
| | to stop after the final | gcctgggcgcctgcgcccccgttcccggcccaaagcccgccgcccgttggaggc |
| | Sp1 binding site of | cattggctggcctttgcgcacagcggatcgattttccaggtgcggagttcactcttgc |
| | the ATP7B promoter, | cgcggttgcttcctttgggacccacggcgtccggcagccaggcgcagagtccgag |
| | containing a portion | gagggggcagcgcagagcggacccgacgcggcgccgccgggcaccttccccg |
| | of the native ATP7B | caggcggtgggtgagccctgggagctgagtctgcggtccggctctgcgcagctca |
| | 5′ UTR | cctgccctcccgctcccgcacacgcgtgagatcccagtacagtgtcggagcgcac |
| | | cagcgcgaggtggccgagaccgcggaggaggacaggcctccgccctgcggcg |
| | | ccggcacggcagaggacattgtggcactggcacggcagagaacactgtggcacc |
| | | ggcggggccggcagttccagggtgggcactcccagccacctggggagtgggcg |
| | | agggtccgaggcccactctcccctcacgctctcatccccgtgcccccaggtcggga |
| | | ggacggcggcgcgcaactttgaatcatccgtgtgaagagggctgcggcttccccg |
| | | gtcccaaatgaaggggcggtt |
| 2 | Sequence of a shorter | cacgaccggctgctcacctcaacaacttgcacaggcaccagctcctttcgccggcc |
| | ATP7B promoter | gccatcttccgccgacccccgaactcaggaaacgcttcactttccttttccctattggc |
| | without negative | tcctgagaaagcaagccgtgctcgccccgcccccacgggccaattgtgcgttacta |
| | regulatory element in | ttggttactggtagccgcttcccacggccttccagccaatagaatatgccgaggcgt |
| | the promoter; This | agactagtgttcggcgtggcgcacacggctcccgcccccgtgggcgggacagca |
| | deletes a metal | gtggggggttgggctgaggagggcgtggcctgtgattgacagccgtcgctccctc |
| | response element-like | cctcggccacctcccccactagaagcccccgcctgggcgcctgcgcccccgttcc |
| | sequence that is | cggcccaaagcccgccgcccgttggaggccattggctggcctttgcgcacagcgg |
| | inhibitor to | atcgattttccaggtgcggagttcactcttgccgcggttgcttcctttgggacccacgg |
| | expression in the | cgtccggcagccaggcgcagagtccgaggagggggcagcgcagagcggaccc |
| | native context | gacgcggcgccgccgggcaccttccccgcaggcggtgggtgagccctgggagc |
| | | tgagtctgcggtccggctctgcgcagctcacctgccctcccgctcccgcacacgcg |
| | | tgagatcccagtacagtgtcggagcgcaccagcgcgaggtggccgagaccgcgg |
| | | aggaggacaggcctccgccctgcggcgccggcacggcagaggacattgtggca |
| | | ctggcacggcagagaacactgtggcaccggcggggccggcagttccagggtgg |
| | | gcactcccagccacctggggagtgggcgagggtccgaggcccactctcccctcac |
| | | gctctcatccccgtgcccccaggtcgggaggacggcggcgcgcaactttgaatcat |
| | | ccgtgtgaagagggctgcggcttccccggtcccaaatgaaggggcggtt |
| 3 | Enhancer element | cacgaccggctgctcacctcaacaacttgcacaggcaccagctcctttcgccggcc |
| | within the ATP7B | gccatcttccgccgacccccgaactcaggaaacgcttcactttccttttccctattggc |
| | promoter | tcctgagaaagcaagccgtgctcgccccgcccccacgggccaatt |
| 4 | Example of intron | CTCTAAGGTAAATATAAAATTTTTAAGTGTATAAT |
| | that can be inserted | GTGTTAAACTACTGATTCTAATTGTTTCTCTCTTTT |
| | into the 5′ UTR; The | AGATTCCAACCTTTGGAACTGA |
| | SV40 intron is | |
| | depicted | |
| 5 | Sequence of the | MPEQERQITAREGASRKILSKLSLPTRAWEPAMKKSF |
| | ATP7B protein; In | AFDNVGYEGGLDGLGPSSQVATSTVRILGMTCQSCV |
| | underline are the | KSIEDRISNLKGIISMKVSLEQGSATVKYVPSVVCLQQ |
| | amino acid | VCHQIGDMGFEASIAEGKAASWPSRSLPAQEAVVKL |
| | polymorphism that | RVEGMTCQSCVSSIEGKVRKLQGVVRVKVSLSNQEA |
| | yield hyperactive | VITYQPYLIQPEDLRDHVNDMGFEAAIKSKVAPLSLG |
| | function | PIDIERLQSTNPKRPLSSANQNENNSETLGHQGSHVVT |
| | | LQLRIDGMHCKSCVLNIEENIGQLLGVQSIQVSLENK |
| | | TAQVKYDPSCTSPVALQRAIEALPPGNFKVSLPDGAE |
| | | GSGTDHRSSSSHSPGSPPRNQVQGTCSTTLIAIAGMTC |
| | | ASCVHSIEGMISQLEGVQQISVSLAEGTATVLYNPAVI |
| | | SPEELRAAIEDMGFEASVVSESCSTNPLGNHSAGNSM |
| | | VQTTDGTPTSLQEVAPHTGRLPANHAPDILAKSPQST |
| | | RAVAPQKCFLQIKGMTCASCVSNIERNLQKEAGVLS |
| | | VLVALMAGKAEIKYDPEVIQPLEIAQFIQDLGFEAAV |
| | | MEDYAGSDGNIELTITGMTCASCVHNIESKLTRINGI |
| | | TYASVALATSKALVKFDPEIIGPRDIIKIIEEIGFHASLA |
| | | QRNPNAHHLDHKMEIKQWKKSFLCSLVFGIPVMAL |
| | | MIYMLIPSNEPHQSMVLDHNIIPGLSILNLIFFILCTFVQ |
| | | LLGGWYFYVQAYKSLRHRSANMDVLIVLATSIAYVY |
| | | SLVILVVAVAEKAERSPVTFFDTPPMLFVFIALGRWL |
| | | EHLAKSKTSEALAKLMSLQATEATVVTLGEDNLIIRE |
| | | EQVPMELVQRGDIVKVVPGGKFPVDGKVLEGNTMA |
| | | DESLITGEAMPVTKKPGSTVIAGSINAHGSVLIKATHV |
| | | GNDTTLAQIVKLVEEAQMSKAPIQQLADRFSGYFVPF |
| | | IIIMSTLTLVVWIVIGFIDFGVVQRYFPNPNKHISQTEV |
| | | IIRFAFQTSITVLCIACPCSLGLATPTAVMVGTGVAAQ |
| | | NGILIKGGKPLEMAHKIKTVMFDKTGTITHGVPRVM |
| | | RVLLLGDVATLPLRKVLAVVGTAEASSEHPLGVAVT |
| | | KYCKEELGTETLGYCTDFQAVPGCGIGCKVSNVEGIL |
| | | AHSERPLSAPASHLNEAGSLPAEKDAAPQTFSVLIGN |
| | | REWLRRNGLTISSDVSDAMTDHEMKGQTAILVAIDG |
| | | VLCGMIAIADAVKQEAALAVHTLQSMGVDVVLITGD |
| | | NRKTARAIATQVGINKVFAEVLPSHKVAKVQELQNK |
| | | GKKVAMVGDGVNDSPALAQADMGVAIGTGTDVAIE |
| | | AADVVLIRNDLLDVVASIHLSKRTVRRIRINLVLALIY |
| | | NLVGIPIAAGVFMPIGIVLQPWMGSAAMAASSVSVVL |
| | | SSLQLKCYKKPDLERYEAQAHGHMKPLTASQVSVHI |
| | | GMDDRWRDSPRATPWDQVSYVSQVSLSSLTSDKPSR |
| | | HSAAADDDGDKWSLLLNGRDEEQYI |
| 6 | Sequence with the | MPEQERQITAREGASRKILSKLSLPTRAWEPAMKKSF |
| | ATP7B, C9 tag; | AFDNVGYEGGLDGLGPSSQVATSTVRILGMTCQSCV |
| | Amino acid residues | KSIEDRISNLKGIISMKVSLEQGSATVKYVPSVVCLQQ |
| | conferring increased | VCHQIGDMGFEASIAEGKAASWPSRSLPAQEAVVKL |
| | activity for ATP7B | RVEGMTCQSCVSSIEGKVRKLQGVVRVKVSLSNQEA |
| | are indicated in bold; | VITYQPYLIQPEDLRDHVNDMGFEAAIKSKVAPLSLG |
| | sequence of the | PIDIERLQSTNPKRPLSSANQNFNNSETLGHQGSHVVT |
| | protein tag is | LQLRIDGMHCKSCVLNIEENIGQLLGVQSIQVSLENK |
| | indicated in | TAQVKYDPSCTSPVALQRAIEALPPGNFKVSLPDGAE |
| | underlining | GSGTDHRSSSSHSPGSPPRNQVQGTCSTTLIAIAGMTC |
| | | ASCVHSIEGMISQLEGVQQISVSLAEGTATVLYNPAVI |
| | | SPEELRAAIEDMGFEASVVSESCSTNPLGNHSAGNSM |
| | | VQTTDGTPTSLQEVAPHTGRLPANHAPDILAKSPQST |
| | | RAVAPQKCFLQIKGMTCASCVSNIERNLQKEAGVLS |
| | | VLVALMAGKAEIKYDPEVIQPLEIAQFIQDLGFEAAV |
| | | MEDYAGSDGNIELTITGMTCASCVHNIESKLTRINGI |
| | | TYASVALATSKALVKFDPEIIGPRDIIKIIEEIGFHASLA |
| | | QRNPNAHHLDHKMEIKQWKKSFLCSLVFGIPVMAL |
| | | MIYMLIPSNEPHQSMVLDHNIIPGLSILNLIFFILCTFVQ |
| | | LLGGWYFYVQAYKSLRHRSANMDVLIVLATSIAYVY |
| | | SLVILVVAVAEKAERSPVTFFDTPPMLFVFIALGRWL |
| | | EHLAKSKTSEALAKLMSLQATEATVVTLGEDNLIIRE |
| | | EQVPMELVQRGDIVKVVPGGKFPVDGKVLEGNTMA |
| | | DESLITGEAMPVTKKPGSTVIAGSINAHGSVLIKATHV |
| | | GNDTTLAQIVKLVEEAQMSKAPIQQLADRFSGYFVPF |
| | | IIIMSTLTLVVWIVIGFIDFGVVQRYFPNPNKHISQTEV |
| | | IIRFAFQTSITVLCIACPCSLGLATPTAVMVGTGVAAQ |
| | | NGILIKGGKPLEMAHKIKTVMFDKTGTITHGVPRVM |
| | | RVLLLGDVATLPLRKVLAVVGTAEASSEHPLGVAVT |
| | | KYCKEELGTETLGYCTDFQAVPGCGIGCKVSNVEGIL |
| | | AHSERPLSAPASHLNEAGSLPAEKDAAPQTFSVLIGN |
| | | REWLRRNGLTISSDVSDAMTDHEMKGQTAILVAIDG |
| | | VLCGMIAIADAVKQEAALAVHTLQSMGVDVVLITGD |
| | | NRKTARAIATQVGINKVFAEVLPSHKVAKVQELQNK |
| | | GKKVAMVGDGVNDSPALAQADMGVAIGTGTDVAIE |
| | | AADVVLIRNDLLDVVASIHLSKRTVRRIRINLVLALIY |
| | | NLVGIPIAAGVFMPIGIVLQPWMGSAAMAASSVSVVL |
| | | SSLQLKCYKKPDLERYEAQAHGHMKPLTASQVSVHI |
| | | GMDDRWRDSPRATPWDQVSYVSQVSLSSLTSDKPSR |
| | | HSAAADDDGDKWSLLLNGRDEEQYITETSQVAPA |
| 7 | Example of sequence | ATGCTTTATTTGTGAAATTTGTGATGCTATTGCTTT |
| | of different | ATTTGTAACCATTATAAGCTGCAATAAACAAGTTA |
| | polyadenylation sites- | ACAACAACAATTGCATTCATTTTATGTTTCAGGTTC |
| | SV40 | AGGGGGAGGTGTGGGAGGTTTTTTAAAGCAAGTAA |
| | polyadenylation | AACCTCTACAAATGTGGTAAAATCCGATAAGGATC |
| | sequence | GATCCGGGC |
| 8 | Example of | AGTCAATATGTTCACCCCAAAAAAGCTGTTTGTTA |
| | sequences of the | ACTTGC |
| | S/MAR element. The | CAACCTCATTCTAAAATGTATATAGAAGCCCAAAA |
| | interferon-beta | GACAATAACAAAAATATTCTTGTAGAACAAAATGG |
| | scaffold/matrix | GAAAGAATGTTCCACTAAATATCAAGATTTAGAGC |
| | attachment region | AAAGCATGAGATGTGTGGGGATAGACAGTGAGGC |
| | | TGATAAAATAGAGTAGAGCTCAGAAACAGACCCA |
| | | TTGATATATGTAAGTGACCTATGAAAAAAATATGG |
| | | CATTTTACAATGGGAAAATGATGGTCTTTTTCTTTT |
| | | TTAGAAAAACAGGGAAATATATTTATATGTAAAAA |
| | | ATAAAAGGGAACCCATATGTCATACCATACACACA |
| | | AAAAAATTCCAGTGAATTATAAGTCTAAATGGAGA |
| | | AGGCAAAACTTTAAATCTTTTAGAAAATAATATAG |
| | | AAGCATGCCATCAAGACTTCAGTGTAGAGAAAAAT |
| | | TTCTTATGACTCAAAGTCCTAACCACAAAGAAAAG |
| | | ATTGTTAATTAGATTGCATGAATATTAAGACTTATT |
| | | TTTAAAATTAAAAAACCATTAAGAAAAGTCAGGCC |
| | | ATAGAATGACAGAAAATATTTGCAACACCCCAGTA |
| | | AAGAGAATTGTAATATGCAGATTATAAAAAGAAGT |
| | | CTTACAAATCAGTAAAAAATAAAACTAGACAAAA |
| | | ATTTGAACAGATGAAAGAGAAACTCTAAATAATCA |
| | | TTACACATGAGAAACTCAATCTCAGAAATCAGAGA |
| | | ACTATCATTGCATATACACTAAATTAGAGAAATAT |
| | | TAAAAGGCTAAGTAACATCTGTGGC |
| 9 | Example of | CATCACATTTAAAAGCATCTCAGCCTACCATGAGA |
| | stabilizing 3′UTR- | ATAAGAGAAAGAAAATGAAGATCAAAAGCTTATT |
| | example of albumin | CATCTGTTTTTCTTTTTCGTTGGTGTAAAGCCAACA |
| | sequence | CCCTGTCTAAAAAACATAAATTTCTTTAATCATTTT |
| | | GCCTCTTTTCTCTGTGCTTCAATTAATAAAAAATGG |
| | | AAAGAATCT |
| 10 | Example of Liver- | CCCTAAAATGGGCAAACATTGCAAGCAGCAAACA |
| | specific promoter- | GCAAACACACAGCCCTCCCTGCCTGCTGACCTTGG |
| | hAPO-HCR | AGCTGGGGCAGAGGTCAGAGACCTCTCTGGGCCCA |
| | Enhancer/hAAT | TGCCACCTCCAACATCCACTCGACCCCTTGGAATTT |
| | promoter | CGGTGGAGAGGAGCAGAGGTTGTCCTGGCGTGGTT |
| | | TAGGTAGTGTGAGAGGGGAATGACTCCTTTCGGTA |
| | | AGTGCAGTGGAAGCTGTACACTGCCCAGGCAAAGC |
| | | GTCCGGGCAGCGTAGGCGGGCGACTCAGATCCCAG |
| | | CCAGTGGACTTAGCCCCTGTTTGCTCCTCCGATAAC |
| | | TGGGGTGACCTTGGTTAATATTCACCAGCAGCCTC |
| | | CCCCGTTGCCCCTCTGGATCCACTGCTTAAATACGG |
| | | ACGAGGACAGG |

### Methods

Disclosed herein are methods of expressing a nucleotide sequence in a specific cell. In some aspects, the specific cell can be a liver cell. In some aspects, the methods can comprise introducing any of the nucleic acid constructs disclosed herein to the specific cell. In some aspects, the specific cell can be a eukaryotic cell. In some aspects, the eukaryotic cell can be a mammalian cell. In some aspects the specific cell can be a specific eukaryotic cell. In some aspects, the specific eukaryotic cell can be a specific mammalian cell. In some aspects, the specific cell can be a diseased cell. In some aspects, the eukaryotic cell can be a diseased cell. In some aspects, the mammalian cell can be a diseased cell.

Disclosed herein are methods of treating a human patient. In some aspects, the methods can comprise administering any of the nucleic acid constructs described herein. In some aspects, the human patient has been identified as being in need of treatment before the administration step. In some aspects, the human patient can have a disease or a disorder. In some aspects, the disease or disorder can be Wilson's disease.

Disclosed herein are methods of delivering any of the nucleic acid constructs described herein to a subject. In some aspects, the methods can comprise administering to the subject an effective amount of a pharmaceutical composition comprising any of the nucleic acid constructs described herein, any of the vector described herein or any of the pharmaceutical compositions described herein.

Disclosed herein are methods of treating Wilson's disease in a subject. In some aspects, the methods can comprise administering any of the nucleic acid constructs described herein, any of the vectors described herein or any of the pharmaceutical compositions described herein to the subject.

Disclosed herein are methods of reducing liver injury in a subject with Wilson's disease. In some aspects, the methods can comprise administering any of the nucleic acid constructs described herein, any of the vectors described herein or any of the pharmaceutical compositions described herein to the subject.

Disclosed herein are methods of reducing hepatic copper levels in a subject with Wilson's disease. In some aspects, the methods can comprise administering any of the nucleic acid constructs described herein, any of the vectors described herein or any of the pharmaceutical compositions described herein to the subject.

Disclosed herein are methods of reducing ALT, AST, or LDH in a subject with Wilson's disease. In some aspects, the methods can comprise administering any of the nucleic acid constructs described herein, any of the vectors described herein or any of the pharmaceutical compositions described herein to the subject.

Also disclosed are methods of delivering any of the nucleic acid constructs described herein, any of the vectors described herein or any of the pharmaceutical compositions described herein to one or more cells. In some aspects, the methods comprise contacting the one or more cells with any of the nucleic acid constructs described herein, any of the vectors described herein or any of the pharmaceutical compositions described herein. In some aspects, the one or more cells can be a specific cell. In some aspects, the specific cell can be a eukaryotic cell. In some aspects, the eukaryotic cell can be a mammalian cell. In some aspects, the specific cell can be a specific eukaryotic cell. In some aspects, the specific eukaryotic cell can be a specific mammalian cell. In some aspects, the cell can be a liver cell. In some aspects, the specific cell can be a diseased cell. In some aspects, the eukaryotic cell can be a diseased cell. In some aspects, the mammalian cell can be a diseased cell.

In some aspects, the subject has been identified as being in need of treatment before the administration step. In some aspects, the subject can have a disease or a disorder. In some aspects, the disease or disorder can be Wilson's disease. In some aspects, the subject can be a human.

In any of the methods disclosed herein, the nucleic acid constructs, the vectors, or the pharmaceutical compositions can be administered to the liver of a subject by hydrodynamic injection. In some aspects, the hydrodynamic injection can a vascular-mediated hydrodynamic injection or a biliary hydrodynamic injection.

The term “treatment” as used herein in the context of treating a disease or disorder, can relate generally to treatment and therapy of a human subject or patient, in which some desired therapeutic effect is achieved, for example, the inhibition of the progress of the disease or disorder, and can include a reduction in the rate of progress, a halt in the rate of progress, regression of the disease or disorder, amelioration of the disease or disorder, and cure of the disease or disorder. Treatment as a prophylactic measure (i.e., prophylaxis, prevention) is also included.

The term “therapeutically-effective amount” as used herein, refers to the amount of the nucleic acid construct that is effective for producing some desired therapeutic effect, commensurate with a reasonable benefit/risk ratio, when administered in accordance with a desired treatment regimen.

Similarly, the term “prophylactically effective amount,” as used herein refers to the amount of the nucleic acid construct that is effective for producing some desired prophylactic effect, commensurate with a reasonable benefit/risk ratio, when administered in accordance with a desired treatment regimen. “Prophylaxis” as used herein refers to a measure which is administered in advance of detection of a symptomatic condition, disease or disorder with the aim of preserving health by helping to delay, mitigate or avoid that particular condition, disease or disorder.

The term “pharmaceutically acceptable,” as used herein, relates to compounds, ingredients, materials, compositions, dosage forms, etc., which are, within the scope of sound medical judgment, suitable for use in contact with the tissues of the subject (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some aspects, the nucleic acid constructs can be formulated to be delivered to cells and organisms in vitro and in vivo in a manner that allows the nucleic acid constructs to carry out their desired biological function. Delivery of nucleic acid constructs can be achieved through the use of non-viral vectors. In some aspects, delivery of nucleic acid constructs can be achieved through without the use of viral vectors. In some aspects, the nucleic acid constructs disclosed herein can be transfected into cells complexed with cationic lipids as well as a variety of other molecules.

In some aspects, the disclosed methods or compositions can be combined with other therapies, whether symptomatic or disease modifying.

The term “treatment” includes combination treatments and therapies, in which two or more treatments or therapies are combined, for example, sequentially or simultaneously. For example it may be beneficial to combine treatment with a compound as described herein with one or more other (e.g., 1, 2, 3, 4) agents or therapies. Appropriate examples of co-therapeutics are known to those skilled in the art based one the disclosure herein. Typically the co-therapeutic can be any known in the art which it is believed may give therapeutic effect in treating the diseases or disorders described herein, subject to the diagnosis of the individual being treated. The particular combination would be at the discretion of the physician who would also select dosages using his/her common general knowledge and dosing regimens known to a skilled practitioner.

The agents (e.g., a disclosed nucleic acid construct (or other therapeutic agent depending on the disorder or disease to be treated), plus one or more other agents) can be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

Disclosed herein are methods of treating a patient. In some aspects, the patient can be in need of any of the nucleic acid constructs disclosed herein.

### Pharmaceutical Compositions

Disclosed herein, are pharmaceutical compositions comprising the nucleic acid constructs described herein. Also, disclosed herein, are pharmaceutical compositions comprising any of the vectors described herein. Further, disclosed herein, are pharmaceutical compositions comprising any of the cells described herein. In some aspects, any of the pharmaceutical compositions disclosed herein can further comprise a pharmaceutical acceptable carrier. The compositions of the present disclosure also contain a therapeutically effective amount of a nucleic acid construct as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. In some aspects, the pharmaceutically acceptable carrier can comprise a lipid-based colloid. In some aspects, the colloid can be a liposome or a lipid nanoparticle.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the pharmaceutical compositions. Thus, compositions can be prepared for parenteral administration that includes nucleic acid constructs dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of condition disorder or disease.

The nucleic acid constructs described herein can be administered to the subject (e.g., a human patient) in an amount sufficient to delay, reduce, or preferably prevent the onset of clinical disease. Accordingly, in some aspects, the patient can be a human patient. In some aspects, the subject can be a human. In therapeutic applications, compositions are administered to a subject (e.g., a human patient) already with or diagnosed with a condition, disorder or disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of the nucleic acid constructs described herein can be an amount that achieves a cure, but that outcome is only one among several that can be achieved. One or more of the symptoms can be less severe. Recovery can be accelerated in an individual who has been treated.

The therapeutically effective amount of the nucleic acid constructs (and any additional therapeutic agent(s) to be combined with the nucleic acid constructs) described herein and used in the methods as disclosed herein applied to mammals (e.g., humans) can be determined by one of ordinary skill in the art with consideration of individual differences in age, weight, and other general conditions (as mentioned above).

Any of the compositions disclosed herein including the nucleic acid constructs described herein can be formulated for administration by any of a variety of routes of administration. In some aspects, any of the nucleic acid constructs or any of the vectors disclosed herein can be administered to the liver of the subject by hydrodynamic injection. In some aspects, the hydrodynamic injection can a vascular-mediated hydrodynamic injection or a biliary hydrodynamic injection.

### EXAMPLES

### Example 1: Functional Expression of the Full-Length Human ATP7B in a Mouse and Pig Livers Using Non-Viral Hydrodynamic Gene Delivery

Wilson disease (WD) is a devastating genetic condition caused by the buildup of copper in multiple tissues, especially in the liver. WD is caused by mutations in ATP7B, which encodes a copper transporter. While gene therapy is a promising approach for treating WD, the ˜4.4 kb ATP7B coding DNA is too large to fit completely into available AAV vectors. To overcome this limitation, a new ATP7B gene therapy strategy was explored using plasmid DNA (pDNA). This approach avoids DNA size restriction, is significantly cheaper than viral vectors, and could be repeated during a patient's lifetime. pDNA encoding human C9-tagged ATP7B (hATP7B) was generated and its expression, localization, and Cu transport activity in vitro was verified prior to injection into animals. Hydrodynamic tail vein injection of Atp7b−/− mice with ATP7B,C9 pDNA under a liver-specific promoter resulted in expression of hATP7B,C9 in hepatocytes and a decrease of hepatic copper levels. To evaluate a possibility of clinical translation, biliary hydrodynamic injection was employed via endoscopic retrograde cholangiography to deliver hATP7B,C9 pDNA into pigs of 40-50 kg in size. A transfection efficiency of 20-30% of hepatocytes was achieved, with positive cells distributed across the zones of the liver lobule and intracellular localization resembling native ATP7B. With the successful demonstration of delivery of the full-sized ATP7B gene into human-sized animals, the evidence described herein show that the pDNA approach can be used to treat WD in patients.

Materials and Methods. Plasmid construction. A human ATP7B (hATP7B) cDNA was utilized that contained polymorphisms at location K832 and R952, which were found to mediate increase copper transporting activity (McCann C J, et al. Metallomics. 2019; 11:1128-1139). The carboxy-terminus tag corresponding to the 9 terminal acid amino residues of the bovine rhodopsin gene (TETSQVAPA; SEQ ID NO: 11) was added by PCR cloning onto the hATP7B gene. The hATP7B,C9 gene was inserted into the pT-LP1-hFIX vector, removing the hFIX gene via XbaI and BglII restriction sites. This vector was constructed through gene synthesis (Bio Basic). The LP1 promoter is derived from a composite of the human apolipoprotein hepatic control region and the human alpha-1-antitrypsin (hAAT) gene promoter (Nathwani A C, et al. Blood. 2006; 107:2653-2661), while the entire expression cassette is located between piggyBac transposon terminal repeats to facilitate integration (Wilson M H, et al. Mol Ther. 2007; 15:139-145). The plasmid, pCMV-hyPBase, was synthesized encoding a hyperactive piggy Bac transposase to facilitate gene integration (Doherty J E, et al. Human Gene Therapy. 2012; 23:311-320). DNA was prepared for injection using QIAgen Plasmid Maxi prep kit for mouse injections and ZymoPURE™ II Plasmid Gigaprep Kit (Zymo Research) for pig injections.

In vitro copper loading assay. Menkes disease fibroblast (YST) cells, lacking active ATP7A and ATP7B, were seeded in 8-well chamber slides at a density of 0.01×10⁶ cells per well. The next day, cells were co-transfected with 100 ng each of either pTyr plasmid alone or with either wt ATP7B plasmid or D1027A GFP-ATP7B (inactive mutant). Twenty hours after transfection, expression of ATP7B was confirmed by GFP signal in DA samples. The cells were then fixed and incubated with L-DOPA for 3 h, then mounted and imaged by phase contrast microscopy to detect formation of eumelanin pigment. Eumelanin pigment formation indicates successful transfer of copper to tyrosinase, which is otherwise depending on copper for enzymatic function.

In vitro ATP7B movement assays. YST cells were seeded at a density of 8×10³ cells per well in 8-well chamber slides in complete media (CM: DMEM, 10% FBS, 1% penicillin/streptomycin). After 48 h, the cells were transfected with 200 ng of C9-ATP7B plasmid using Lipofectamine LTX and Plus reagent system. After 20 h, the media was changed to either basal (CM with 9 mg/ml cycloheximide), high Cu (basal+100 μM Cu), or low Cu (basal+25 μM TTM) media and incubated for 3 h at 37° C. The cells were washed with PBS, fixed using 4% PFA for 15 min, permeabilized with 0.1% Triton X-100 for 15 min, and blocked with 5% BSA for 40 min at RT. After washing with PBS, the cells were incubated with primary antibodies in PBS with 0.1% Triton X-100 (mouse 1D4 at 1:400 or rabbit anti-7B at 1:400, sheep TGN46 at 1:600) for 1 h at RT. The cells were washed twice with PBST for 5 min and once with PBS for 5 min. The cells were then incubated with secondary antibodies in PBS with 0.1% Triton X-100 (anti-mouse Alexa488 at 1:400 or anti-rabbit Alexa 488 at 1:1000, anti-sheep Alexa 555 at 1:1000) for 1 h at RT, protected from light. The cells were washed twice with PBST for 5 min and once with PBS for 5 min, dried, mounted using Fluoromount-DAPI, and cured in the dark. The cells were imaged using LSM 800 confocal microscope with 63× oil len.

Mouse experiments. A C57BL6 mouse model with a null ATP7B mutation was utilized in the experiments (Muchenditsi A, et al. Scientific Reports. 2021; 11:5659-16). Mouse were bred with homozygous AT7B KO males crossed with ATP7B heterozygous females. This led to the generation of heterozygous and homozygous littermates that were utilized in the experiments.

Mice were injected under established protocols for hydrodynamic tail vein injection (Liu F, et al. Gene Therapy. 1999; 6:1258-1266). Briefly, mice were warmed under heat lamp to induce vasodilation of their lateral tail veins. Using a 27 gauge needle, pDNA in saline solution corresponding to 10% of the body weight was injected into the mice within 5-7 seconds. Different amounts of pT-LP1-ATP7B,C9 and pCMV-hyPBase were injected depending on experiment. In the low-dose cohort, 1 μg pT-LP1-ATP7B,C9 and 1 μg pCMV-hyPBase were injected. In the middle dose cohort, 5 μg pT-LP1-ATP7B, C9 and 5 μg pCMV-hyPBase were injected. In the high dose cohort, 25 μg pT-LP1-ATP7B,C9 and 10 μg pCMV-hyPBase were injected.

Serum was obtained by retro-orbital bleed from mice, and chemistries analyzed by the Johns Hopkins Phenotyping Core. Serum with noted hemolysis were excluded from analysis for AST and LDH. Mice were euthanized and perfused with saline before liver harvest and tissue analysis.

Biliary hydrodynamic injection. A detailed protocol for biliary hydrodynamic injection was used (Kumbhari V, et al. Gastrointest. Endosc. 2018; 88:755-763.e5; and Huang Y, et al. PLOS ONE. 2021; 16:e0249931). Briefly, pigs were anesthetized for the procedure and monitored throughout for heart rate, blood pressure, and ventilation. An endoscope was advanced through the mouth and eventually into the small intestine. A catheter was next advanced through the ampulla of Vater into the common bile duct. A balloon on the catheter was subsequently inflated within the common hepatic duct. The catheter was connected to a power injector, and the pigs were injected with pDNA dissolved in saline solution using a power injector at parameters of 40 mL volume at 4 mL/second. Blood draws were collected before and after hydrodynamic in pigs to monitor liver toxicity.

Immunostaining. For both mouse and pig studies, the use of a C9-tag was leveraged to distinguish delivered hATP7B from host mouse and pig ATP7B. To detect the C9-tag, the 1D4 monoclonal antibody clone (mouse, Santa Cruz, Cat #57432), that reacts with the 9 carboxy-terminal amino acids from the bovine rhodopsin protein (C9-tag), was used. A mouse-on-mouse (MOM) protocol was used to reduce background staining in mouse liver. For immunofluorescent staining of pig tissue, a polyclonal ATP7B antibody (ThermoFisher Scientific) was utilized with FITC labeled secondary antibody, while 1D4 antibody was utilized with the secondary antibody, goat anti-Mouse Alexa 647. For quantification of mouse transfection efficiency from hydrodynamic injection, individual hepatocytes were counted in random fields using ImageJ (with at least 5 fields counted at 20× per mouse). For quantification of pig transfection efficiency, the immunostained area within individual hepatic lobules, easily demarcated by fibrous tissue was quantified using ImageJ.

Copper measurements. Hepatic copper measurements were obtained.

Statistics. Unpaired, parametric t-tests were used to determine statistical significance (p<0.05). Statistical analyses were performed using GraphPad Prism 9.0.0 for Mac, GraphPad Software, San Diego, California USA, www.graphpad.com.

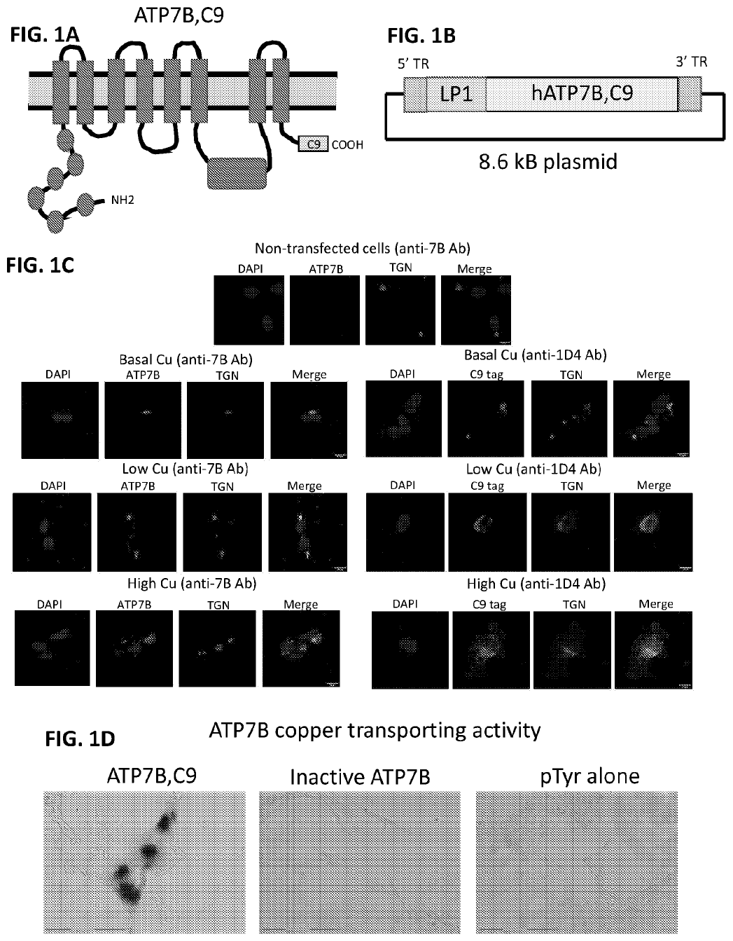

Results. Validation of plasmid DNA vector encoding human ATP7B, C9 in vitro. A pDNA vector was first constructed to efficiently express hATP7B after delivery. Given that the goal was to inject the hATP7B plasmid into wild-type pigs, a c-terminal tag to ATP7B encoding the last nine amino acids from the human rhodopsin protein, C9-tag (FIG. 1A), was added. The C9-tag was located at protein c-terminus but has been used to tag membrane proteins in other studies, with the epitope typically located intracellularly (Molday L L, Molday R S. Methods Mol. Biol. 2014; 1177:1-15). The design of hATP7B,C9 described herein fulfills these features. The hATP7B,C9 gene was cloned into a plasmid DNA vector driven by a liver-specific promoter, with expression cassette located within piggy Bac terminal repeats to mediate integration (FIG. 1B).

While a c-terminal myc tag on ATP7B has been employed without affecting ATP7B function (Zhu M, et al. CNS Neurosci Ther. 2013; 19:346-351), it was sought to validate that the introduced C9-tag would not alter ATP7B localization and function. First, ATP7B,C9 was transfected into YST cells, which lack native ATP7B expression. Immunofluorescent staining demonstrated that ATP7B,C9 can be efficiently detected within anti-C9 antibody, with the staining pattern was indistinguishable from ATP7B (FIG. 1C). Importantly, this staining co-localized with the trans-golgi network (TGN) at basal copper levels, indicating proper ATP7B,C9 localization. Next, it was tested whether ATP7B,C9 retained proper movement inside cells in response to copper. It was observed in high copper conditions that ATP7B, C9 moved out in speckled pattern beyond the TGN in the expected pattern for wildtype ATP7B (FIG. 1C). In low copper conditions, ATP7B,C9 remained in the TGN as expected.

Next, functional assays were performed with ATP7B,C9. Copper transport into the secretory pathway by ATP7B,C9 was monitored by evaluating copper loading into the copper-dependent enzyme tyrosinase (Roy S, et al. Scientific Reports. 2020; 10:13487-15). Formation of eumelanin pigment by tyrosinase can be visually appreciated, indicating successful copper transport. Efficient eumelanin with ATP7B,C9, while an inactive mutant ATP7B lacked any eumelanin, along with transfected cells without any ATP7B was also observed (FIG. 1D).

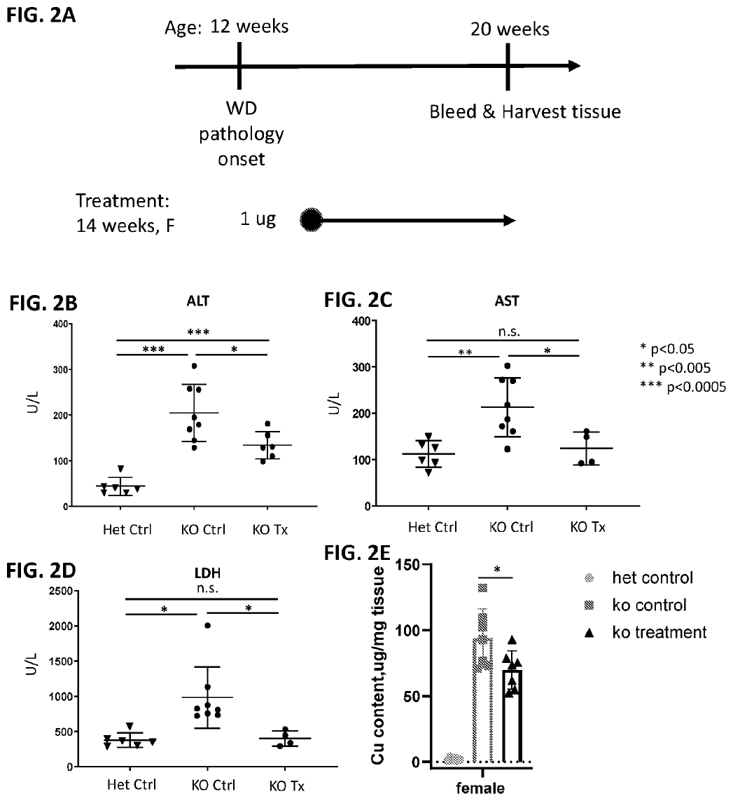

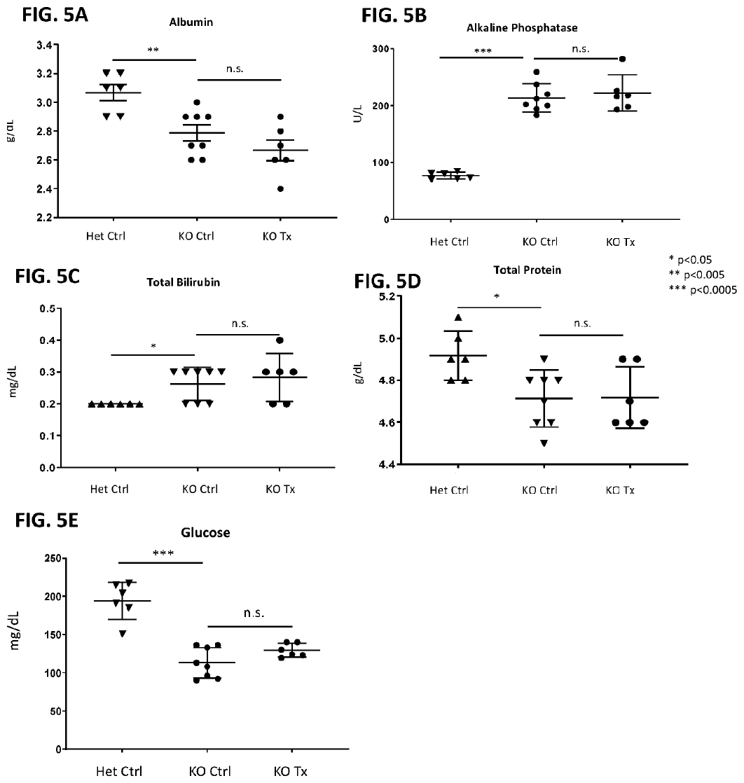

Validation function of ATP7B after hydrodynamic injection in a mouse model of Wilson's disease. Next, the function of ATP7B, C9 after non-viral delivery of pDNA into WD mice was validated. WD mice with pathology already present, which starts around 12 weeks age in C57BL6 mice, were treated. This would show that gene delivery to a WD liver with injury was possible, as most WD patients already have liver phenotype at diagnosis. Also a low dose of pDNA, 1 μg, was employed to assess the impact of fewer hepatocytes transfected, and whether these transfected cells would expand over time with integrated hATP7B,C9. Mice were injected around 14 weeks ago, and evaluated at 20 weeks (FIG. 2A) (Muchenditsi A, et al. Scientific Reports. 2021; 11:5659-16). After 6 weeks of therapy, several markers in the Wilson's disease treated mice were significantly reduced, including ALT, AST, and LDH (FIGS. 2B-D). Other markers (alkaline phosphatase, total bilirubin) did not show a significant difference from the untreated group (FIG. 5). Hepatic copper levels were also reduced by 27% in treated WD mice, indicating successful ATP7B,C9 function in vivo (FIG. 2E).

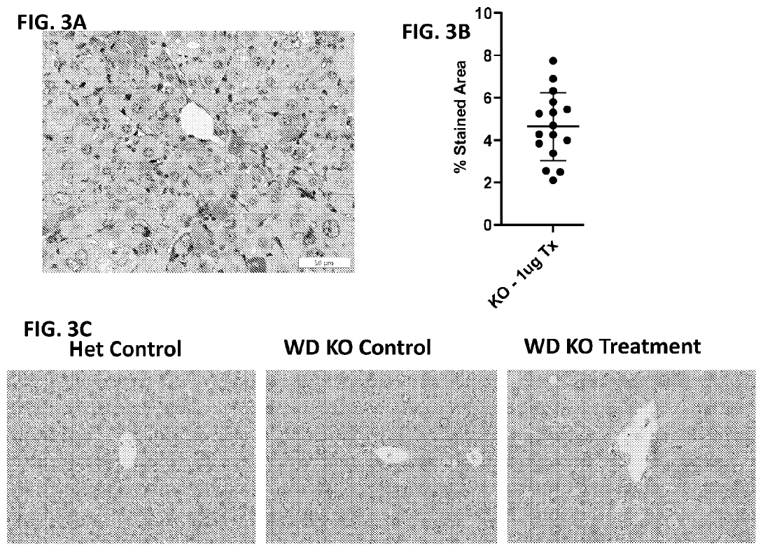

Looking at gene delivery into the liver, the presence of ATP7B, C9 hepatocytes by immunohistochemistry for the C9-tag in the mice harvested at 20 weeks was observed, confirming their presence to mediate the copper reduction (FIG. 3A). Hepatocytes exhibit a range of staining intensities, likely reflecting different expression. The percentage of ATP7B,C9 hepatocytes was calculated to be 4.64% of total hepatocytes in mice injected with 1 μg of plasmid DNA (FIG. 3B). This percentage was compared to heterozygous mice injected with ATP7B,C9, finding similar gene delivery had occurred regardless of liver status (WD or normal), although no relative expansion of ATP7B,C9 in WD mice was observed over this time point (FIG. 3B). The overall histology among the mice did not demonstrate noticeable differences from treated to untreated mice (FIG. 3C), which matches the transaminase elevation still present in treated groups.

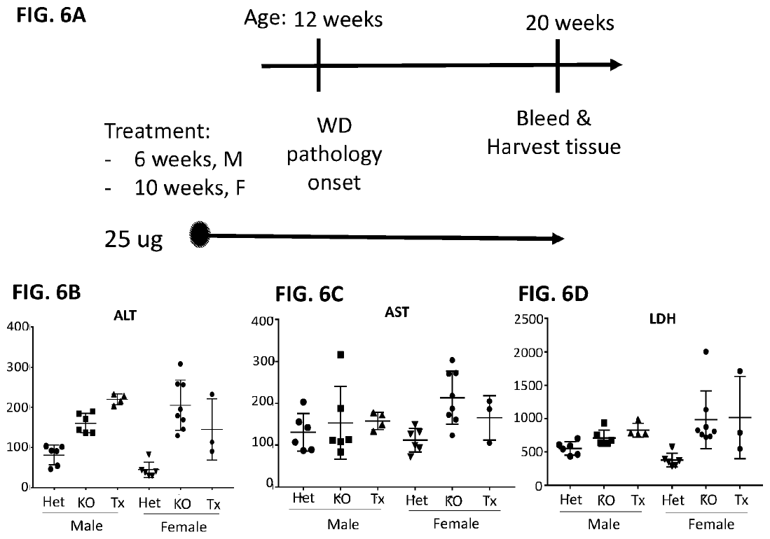

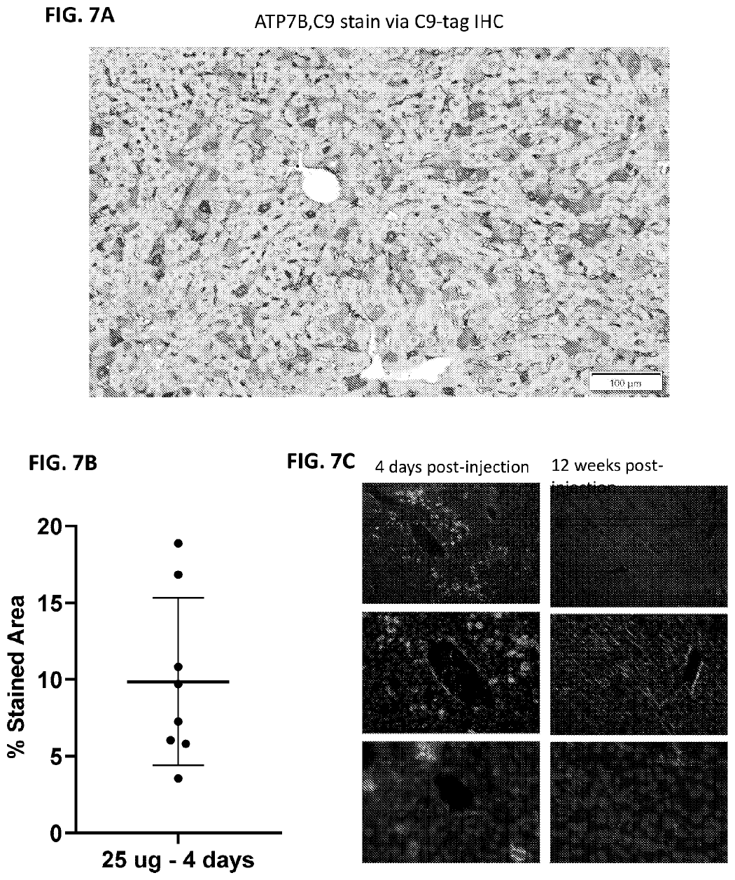

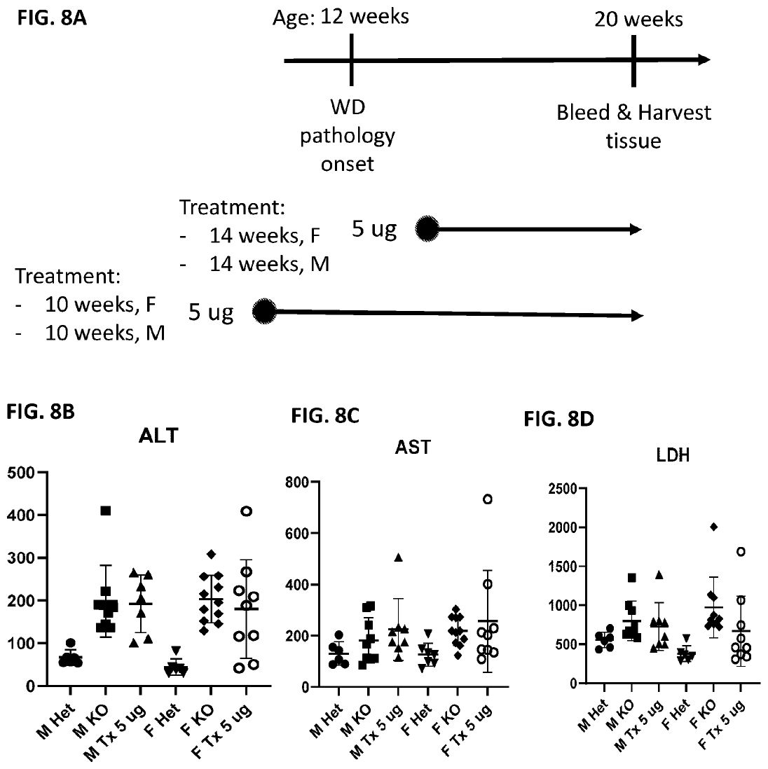

higher doses of pDNA hydrodynamic injection were evaluated to increase transfection percentage. WD mice treated with 25 μg pT-LP1-hATP7B,C9 surprisingly did not exhibit improvements in liver enzymes (FIG. 6), which correlated with the absence of ATP7B,C9 protein on immunofluorescent staining (FIG. 7). This contrasts with 9.86% of hepatocytes transfected 4 days post-injection with 25 μg pDNA (FIG. 7). A reduced dose of 5 μg pT-LP1-hATP7B,C9 had similar results, with lack of improvement in liver transaminases (FIG. 8).

Biliary hydrodynamic injection of ATP7B plasmid in pigs. To treat human patients with WD, it was important to translate hydrodynamic injection of hATP7B,C9 pDNA into a human-sized animal model. The systemic, vascular hydrodynamic tail vein injection in mice is not applicable to human patients. To this end, hydrodynamic injections through the biliary system into pigs, which efficiently branches into the lobes and contacts the hepatocytes was carried out (Kumbhari V, et al. Gastrointest. Endosc. 2018; 88:755-763.e5; and Huang Y, et al. PLOS ONE. 2021; 16: e0249931). In addition, biliary hydrodynamic injection in pigs can achieve a higher transfection efficiency than mouse tail vein injection.

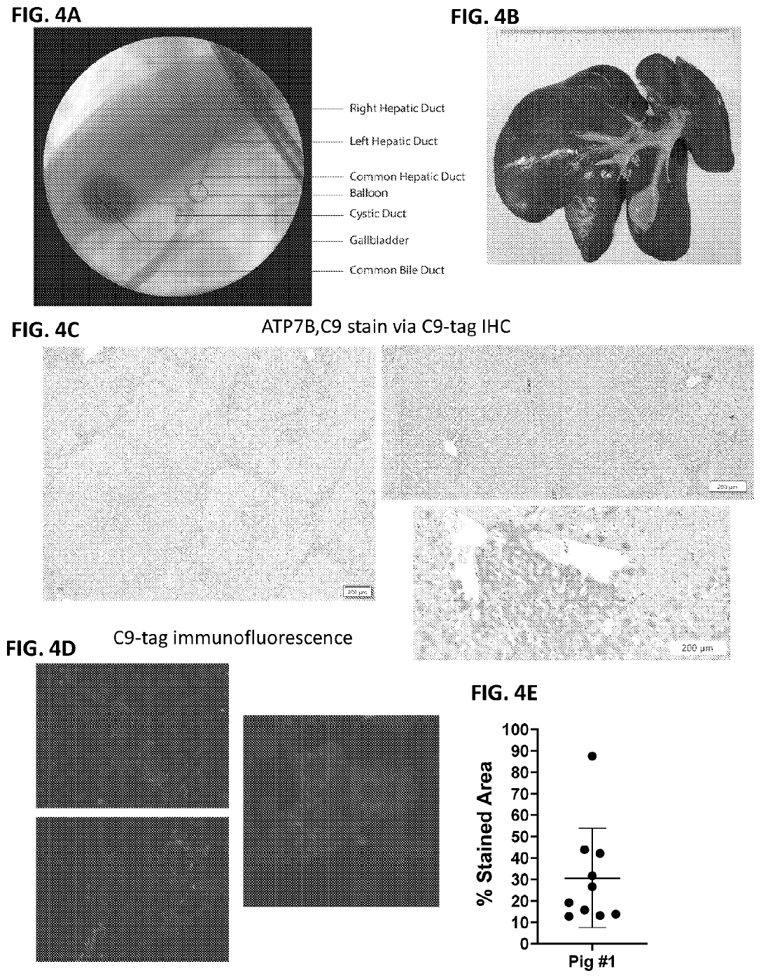

Two pigs weighing between 40-50 kg were obtained for testing hATP7B,C9 gene delivery via endoscopic retrograde cholangio-pancreatography (ERCP). Pigs were monitored before and after injection and demonstrated no elevation in liver transaminases. Fluoroscopy before the injection confirmed catheter placement of the catheter in the common hepatic duct (FIG. 4A). Ten mg of ATP7B,C9 pDNA was injected into the first pig. As shown in FIG. 4B, pig liver was harvested at day 1 post-injection demonstrating no abnormalities or lesions after biliary hydrodynamic injection. DNA was extracted from the lobes, and PCR testing was able to correctly localize ATP7B DNA in the liver lobes including proximal and distal locations in the lobe compared to the injection site. Evaluating for protein expression by immunohistochemistry (IHC), ATP7B,C9 was detected in the pig liver lobes. As shown in FIG. 4C, there were positive ATP7B,C9 staining along the lobular borders as well as near the central vein across the three zones. Immunostained cells were clearly distinguishable from neighboring negative hepatocytes, with some variation in intensity of staining. Interesting, certain lobules appeared to express ATP7B,C9 among almost 80-90% of hepatocytes (FIG. 4C). On total, the average transfection efficiency was 30.58%, while a second pig injected with a lower dose of 5 mg had a transfected efficiency of 15% (FIG. 4E).

The localization of ATP7B,C9 protein in pig hepatocytes was confirmed by immunofluorescence staining (FIG. 4D). ATP7B,C9 was observed to be correctly located in pig hepatocytes, with a pattern distinguishing from endogenous ATP7B in pigs, as well as ATP7B in mouse hepatocytes. The total expression of ATP7B from pig injection was compared with the expression of ATP7B for the mouse tail vein injection by Western blot. Protein expression of ATP7B, C9 was also found to yield approximately 20% of the pigs by Western blot.

Discussion. The results described herein demonstrate the successful gene delivery of hATP7B into a large animal model. It was validated that hATP7B expressed from pDNA is functional in vitro and in WD mouse models, before achieving significant transfection of pig hepatocytes with hATP7B,C9. Given that the pigs used are approximately the size of an adult human female and that a routine clinical procedure employed in ERCP, the approach disclosed herein is translatable to patients with Wilson's disease. These results are an important step in demonstrating gene therapy for this patient population.

Greater than 20% hATP7B gene deliver was achieved into pigs. It is known that other studies of Wilson's disease gene therapy have to find that transfection efficiency greater than 10% could normalize liver histology and injury and mouse models, in that gene delivery greater than 20% could normalize ceruloplasmin levels as well. In an early study, the Long-Evans cinnamon rat model suggested complete correction with 20% of hepatocytes (Irani A N, et al. Mol Ther. 2001; 3:302-309), while a recent study suggested correction above 20% is sufficient to normalize phenotype, with gains in copper reduction observed above 10% (Murillo O, et al. Hepatology. 2019; 70:108-126). These two studies suggest that a smaller number of hepatocytes can act as a sink for copper. The data described herein fall in line with these previous studies, wherein a 4.6% transfection in WD mice yielded a 27% copper reduction, stoichiometric with the 20% threshold that has been published. Together, the delivery efficacy in pigs can fully reverse disease pathology in patients with Wilson's disease.

The results demonstrate that hATP7B can be expressed from a hydrodynamically delivered plasmid vector in mice and pigs. Non-viral hydrodynamic gene delivery of pDNA has no defined size limit for the DNA vector, in comparison to AAV vectors previously employed. Successful expression of an 8.6 kB pDNA in pigs, which is 79% larger than the AAV genome packaging limit, was achieved. The data described herein support the use of full-length hATP7B over the use of truncated miniature ATP7B, which lack several metal binding domains, and have been used in AAV studies.

Importantly, it was observed that hATP7B can be delivered into a diseased WD liver and was functional in WD mice, reducing liver injury and decreasing hepatic copper content. The level of hATP7B hepatocyte transfection (4.6%) was not enough to completely cure the disease. This is consistent with previous WD gene therapy studies, which suggested a level of 20% is important for a cure in a WD mouse model (Murillo O, et al. Hepatology. 2019; 70:108-126). Increasing pDNA doses in mice mildly increased transfection efficiency to 10%, but this appeared to result in overexpression toxicity or immune responses to ATP7B since mice were later negative for ATP7B,C9 staining. As a comparison, hydrodynamic delivery of low density lipoprotein receptor (LDLR) DNA in the treatment of familial hypercholesterolemia in mouse models has yielded toxicity from overexpression with too much lipid accumulation (Cichon G, et al. J. Gene Med. 2004; 6:166-175). As a membrane protein, ATP7B could accumulate inside the cell's TGN eventually disrupting other cell processes. Importantly, a small dose of 1 μg of ATP7B in mice was able to achieve therapeutic benefits despite transfecting very few cells (<4.6%).

An unresolved question in gene therapy for WD is if the corrected hepatocytes have a proliferative advantage compared to the surrounding diseased hepatocytes. Proliferative advantages are notably occur in two other monogenetic liver diseases, hereditary tyrosinemia type I (Hickey R D, et al. Science Translational Medicine. 2016; 8:349ra99-349ra99) and alpha-1 antitrypsin disease (Borel F, et al. Mol Ther. 2017; 25:2477-2489). In these disorders, an initial population of a few percent corrected cells can expand over months to greater than half the liver population. WD may have the same mechanism, given the hepatocyte damage, given ongoing hepatocyte damage. A study of hepatocyte cell therapy observed hepatocyte repopulation over 1 year to almost 100% of cells (Irani A N, et al. Mol Ther. 2001; 3:302-309), but that investigation used chemicals (retrosine) to artificially augment liver damage. As disclosed herein, a mild proliferative advantage of transfected cells over 6 weeks was observed, although it's possible much longer time points may elucidate this further.

In comparing the results disclosed herein to AAV, AAV can be dosed to transduce the majority of hepatocytes in mice, while HTVI is limited transfected at most 20% of hepatocytes, and in many studies 5-10%. ssDNA to dsDNA conversion among AAV genomes can be inefficient, however, leading to lower levels of hepatocytes actually expressing hATP7B, while hydrodynamic injection delivers pDNA that is expressed within hours after injection. This may have resulted in lower net levels of ATP7B expressed across more hepatocytes, contrasting with the data disclosed herein. Moreover, previous studies lacked enhancer elements in the promoter in order to make expression cassette fit in the AAV backbone, further reducing expression.

The results disclosed herein demonstrate gene delivery of ATP7B DNA into a human-sized animal model. Also, the results disclosed herein demonstrate that the plasmid DNA mediated expression of ATP7B creates functional protein in both tissue and mouse models. Given that clinically available equipment and an ERCP procedure used in routine clinical practice were used, the methods described herein can be translated into the treatment of Wilson's disease in patients.

The non-viral approach can be used to redose gene therapy over time, improve the safety of the system, and represent significant cost savings in manufacturing.

What is claimed is:

1. A nucleic acid construct comprising:
a) a promoter;
b) a 5′ untranslated region (5′UTR);
c) a nucleic acid sequence encoding the ATP73 polypeptide, a variant of the ATP73 polypeptide or a polypeptide having at least 90% identity to SEQ ID NO: 5;
d) a 3′ untranslated region (3′UTR); and
e) a polyadenylation sequence.

2. The nucleic acid construct of claim 1, wherein the promoter is a liver-specific promoter.

3. The nucleic acid construct of claim 1, wherein the promoter is constitutively active.

4. The nucleic acid construct of claim 2, wherein the liver-specific promoter is an alpha-1 antitrypsin, human thyroxine binding globulin, hemopexin, albumin, or HBV core promoter.

5. The nucleic acid construct of claim 1, wherein the promoter is a segment of the human ATP7B promoter.

6. The nucleic acid construct of claim 5, wherein promoter is the human metallothionein promoter.

7. The nucleic acid construct of claim 5, wherein promoter is a synthetic promoter, wherein the synthetic promoter comprises one or more MTF1 transcription factor binding sites.

8. The nucleic acid construct of claim 5, wherein the segment of the human ATP7B promoter comprises a negative regulatory element, wherein the negative regulatory element is replaced with an enhancer element.

9. The nucleic acid construct of claim of claim 8, wherein nucleotides −811 to −1265 of the negative regulatory element of the human ATP7B promoter is replaced with the enhancer element.

10. The nucleic acid construct of claim 9, wherein the enhancer element is a liver-specific enhancer element.

11. The nucleic acid construct of claim 10, wherein the liver-specific enhancer element is a human apolipoprotein hepatic control region, human albumin enhancer, human ApoE enhancer, or a viral enhancer.

12. In some aspects, the viral enhancer is a SV40 enhancer, a HBV enhancer I, or a HBV enhancer II.

13. The nucleic acid construct of claim 10, wherein the liver-specific enhancer element comprises a metal responsive element (MRE) site.

14. The nucleic acid construct of claim 13, wherein the MRE site is responsive to copper.

15. The nucleic acid construct of claim 1, wherein the 5′UTR comprises an intron.

16. The nucleic acid construct of claim 15, wherein the intron is a SV40 intron, a Minute Virus of Mice (MVM) intron, or a human growth hormone (HGH) intron.

17. The nucleic acid construct of claim 1, wherein nucleic acid sequence encoding the ATP73 polypeptide is a cDNA encoding full-length ATP73 protein.

18. The nucleic acid construct of claim 1, wherein the nucleic acid sequence encoding the ATP73 polypeptide comprises one or more introns.

19. The nucleic acid construct of claim 1, wherein the nucleic acid sequence encodes a variant of the ATP73 polypeptide comprising a polymorphism at position K832, R952, or a combination thereof.

20. The nucleic acid construct of claim 1, wherein the nucleic acid sequence encoding the ATP73 polypeptide is codon optimized.

21. The nucleic acid construct of claim 1, wherein the nucleic acid sequence encoding the ATP73 polypeptide further comprises a protein tag at the C-terminus.

22. The nucleic acid construct of claim 21, wherein the protein tag is a C9 tag or a c-myc tag.

23. The nucleic acid construct of claim 22, wherein the C9 tag is a 9 terminal acid amino residues of the bovine rhodopsin gene (TETSQVAPA; SEQ ID NO: 11).

24. The nucleic acid construct of claim 1, wherein the 3′UTR is alpha-hemoglobin, beta-hemoglobin, albumin, mitochondrially encoded 12S rRNA (mtRNR1), or amino-terminal enhancer of split (AES).

25. The nucleic acid construct of claim 24, wherein the 3′UTR comprises a scaffold/matrix attachment region (S/MAR) element.

26. The nucleic acid construct of claim 1, wherein the polyadenylation sequence comprises a SV40 polyadenylation sequence, a human growth hormone polyadenylation sequence, a bovine growth hormone polyadenylation sequence, or a rabbit beta-globin polyadenylation sequence.

27. The nucleic acid construct of claim 1, further comprising a bacterial origin of replication and/or a selection system.

28. The nucleic acid construct of claim 27, wherein the bacterial origin of replication comprises a pUC origin, R6K origin, or a miniaturized pUC origin.

29. The nucleic acid construct of claim 27 or 28, wherein the selection system comprises an RNA-OUT, tRNA, cccdB/cccdA, or an antibiotic resistance gene.

30. The nucleic acid construct of claim 29, wherein the antibiotic resistance gene is ampicillin, kanamycin, or zeocin.

31. A vector comprising any of the nucleic acid constructs of claims 1-30.

32. The vector of claim 31, wherein the vector is a plasmid or a bacteriophage.

33. The vector of claim 31, wherein the vector is not a virus.

34. A cell comprising any of the nucleic acid constructs of claims 1-30 or any of the vectors of claims 31-33.

35. A pharmaceutical composition comprising the nucleic acid construct of any of claims 1-30, any of the vectors of claims 31-33 or the cell of claim 34.

36. The pharmaceutical composition of claim 35, further comprising a pharmaceutically acceptable carrier.

37. The pharmaceutical composition of claim 36, wherein the pharmaceutically acceptable carrier comprises a lipid-based colloid.

38. The pharmaceutical composition of claim 37, wherein the colloid is a liposome or a lipid nanoparticle.

39. A method of delivering a nucleic acid construct to a subject, the method comprising administering to the subject an effective amount of a pharmaceutical composition comprising any of the nucleic acid constructs of any of claims 1-30, the vector of any of claims 31-33 or the pharmaceutical compositions of any of claims 35-38.

40. A method of treating Wilson's disease in a subject, the method comprising: administering the nucleic acid construct of any of claims 1-30, the vector of any of claims 31-33 or the pharmaceutical compositions of any of claims 35-38 to the subject.

41. A method of reducing liver injury in a subject with Wilson's disease, the method comprising: administering the nucleic acid construct of any of claims 1-30, the vector of any of claims 31-33 or the pharmaceutical compositions of any of claims 35-38 to the subject.

42. A method of reducing hepatic copper levels in a subject with Wilson's disease, the method comprising: administering the nucleic acid construct of any of claims 1-30, the vector of any of claims 31-33 or the pharmaceutical compositions of any of claims 35-38 to the subject.

43. A method of reducing ALT, AST, or LDH in a subject with Wilson's disease, the method comprising: administering the nucleic acid construct of any of claims 1-30, the vector of any of claims 31-33 or the pharmaceutical compositions of any of claims 35-38 to the subject.

44. The method of any of claims 39-43, wherein the subject has been identified as being in need of treatment.

45. The method of any of claims 39-43, wherein the subject is a human.

46. The method of any of the preceding claims, wherein the nucleic acid construct or the vector or the pharmaceutical composition is administered to the liver of the subject by hydrodynamic injection.

47. The method of claim 46, wherein the hydrodynamic injection is a vascular-mediated hydrodynamic injection or a biliary hydrodynamic injection.


SEQUENCE LISTING (11 sequences)

SEQ ID NO: 1 - DNA, 1070 bp, synthetic construct
  source 1..1070: mol_type = other DNA
acaaggaaggccatttgcccgcaaaatttagctacactggacgggcaagtacccctacagaagagaaaacgtctgtgagcccacacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaattgtgcgttactattggttactggtagccgcttcccacggccttccagccaatagaatatgccgaggcgtagactagtgttcggcgtggcgcacacggctcccgcccccgtgggcgggacagcagtggggggttgggctgaggagggcgtggcctgtgattgacagccgtcgctccctccctcggccacctcccccactagaagcccccgcctgggcgcctgcgcccccgttcccggcccaaagcccgccgcccgttggaggccattggctggcctttgcgcacagcggatcgattttccaggtgcggagttcactcttgccgcggttgcttcctttgggacccacggcgtccggcagccaggcgcagagtccgaggagggggcagcgcagagcggacccgacgcggcgccgccgggcaccttccccgcaggcggtgggtgagccctgggagctgagtctgcggtccggctctgcgcagctcacctgccctcccgctcccgcacacgcgtgagatcccagtacagtgtcggagcgcaccagcgcgaggtggccgagaccgcggaggaggacaggcctccgccctgcggcgccggcacggcagaggacattgtggcactggcacggcagagaacactgtggcaccggcggggccggcagttccagggtgggcactcccagccacctggggagtgggcgagggtccgaggcccactctcccctcacgctctcatccccgtgcccccaggtcgggaggacggcggcgcgcaactttgaatcatccgtgtgaagagggctgcggcttccccggtcccaaatgaaggggcggtt

SEQ ID NO: 2 - DNA, 987 bp, synthetic construct
  source 1..987: mol_type = other DNA
cacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaattgtgcgttactattggttactggtagccgcttcccacggccttccagccaatagaatatgccgaggcgtagactagtgttcggcgtggcgcacacggctcccgcccccgtgggcgggacagcagtggggggttgggctgaggagggcgtggcctgtgattgacagccgtcgctccctccctcggccacctcccccactagaagcccccgcctgggcgcctgcgcccccgttcccggcccaaagcccgccgcccgttggaggccattggctggcctttgcgcacagcggatcgattttccaggtgcggagttcactcttgccgcggttgcttcctttgggacccacggcgtccggcagccaggcgcagagtccgaggagggggcagcgcagagcggacccgacgcggcgccgccgggcaccttccccgcaggcggtgggtgagccctgggagctgagtctgcggtccggctctgcgcagctcacctgccctcccgctcccgcacacgcgtgagatcccagtacagtgtcggagcgcaccagcgcgaggtggccgagaccgcggaggaggacaggcctccgccctgcggcgccggcacggcagaggacattgtggcactggcacggcagagaacactgtggcaccggcggggccggcagttccagggtgggcactcccagccacctggggagtgggcgagggtccgaggcccactctcccctcacgctctcatccccgtgcccccaggtcgggaggacggcggcgcgcaactttgaatcatccgtgtgaagagggctgcggcttccccggtcccaaatgaaggggcggtt

SEQ ID NO: 3 - DNA, 160 bp, synthetic construct
  source 1..160: mol_type = other DNA
cacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaatt

SEQ ID NO: 4 - DNA, 93 bp, synthetic construct
  source 1..93: mol_type = other DNA
ctctaaggtaaatataaaatttttaagtgtataatgtgttaaactactgattctaattgtttctctcttttagattccaacctttggaactga

SEQ ID NO: 5 - protein, 1465 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPAVISPEELRAAIEDMGFEASVVSESCSTNPLGNHSAGNSMVQTTDGTPTSLQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAAPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 6 - protein, 1474 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPAVISPEELRAAIEDMGFEASVVSESCSTNPLGNHSAGNSMVQTTDGTPTSLQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAAPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYITETSQVAPA

SEQ ID NO: 7 - DNA, 186 bp, synthetic construct
  source 1..186: mol_type = other DNA
atgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatccgataaggatcgatccgggc

SEQ ID NO: 8 - DNA, 800 bp, synthetic construct
  source 1..800: mol_type = other DNA
agtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggc

SEQ ID NO: 9 - DNA, 186 bp, synthetic construct
  source 1..186: mol_type = other DNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 10 - DNA, 398 bp, synthetic construct
  source 1..398: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 11 - protein, 9 aa, synthetic construct
TETSQVAPA